(19) United States
(12) Patent Application Publication
(10) Pub. No.: US 2016/0001073 A1
(43) Pub. Date: Jan. 7, 2016

(54) APPARATUS AND METHOD FOR STIMULATING HAIR GROWTH AND/OR PREVENTING HAIR LOSS

(71) Applicant: PILOGICS L.P., Haifa (IL)

(72) Inventors: Dov INGMAN, Herzliya (IL); Erez MANOR, Herzlia (IL)

(21) Appl. No.: 14/739,832

(22) Filed: Jun. 15, 2015

Related U.S. Application Data
(60) Provisional application No. 62/011,705, filed on Jun. 13, 2014.

(51) Int. Cl. A61N 1/36; A61N 1/32; A61N 1/04

(52) U.S. Cl. CPC A61N 1/36014; A61N 1/0464; A61N 1/0476; A61N 1/322

(57) ABSTRACT

A method of treating or preventing a hair-condition of a user comprising: subjecting the user's scalp to at least 200 distinct electrode-scalp contact events during a time-interval of at most one minute and dividable into 5 non-overlapping equal-duration sub-intervals covering the time-interval, method performed such that i. for at least a majority of the electrode-scalp contact events, no electrode of the event enters into the dermis; ii. a duration of each electrode contact event is at most 100 milliseconds; and iii. for each electrode contact event, an electrical current flows between the electrode and the scalp so as to deposit electrode-released ions of a first metal or of a second metal on the scalp, thereby forming a respective metal-ion-deposition island on the user's scalp.

### CROSS-REFERENCE TO RELATED APPLICATIONS

This patent application claims the benefit of U.S. Provisional Patent Application No. 62/011,705, filed on Jun. 13, 2014, which is incorporated herein by reference in its entirety.

Some embodiments of the present invention relate to methods and apparatus that were disclosed in PCT/IB2012/057041 which (i) was filed on Dec. 12, 2012; (ii) was published on Jun. 13, 2013 as WO/2013/084189; and (iii) is incorporated herein by reference in its entirety. In some embodiments, any feature or combination of features described in the present document may be combined with any feature of combination of features described in application PCT/IB2012/057041. In some embodiments, any feature or combination of feature(s) disclosed in application PCT/IB2012/057041 may be modified—e.g. electrode-needles thereof may be configured so they are not as sharp, and/or so that they are non-wounding—e.g. configured not to penetrate (e.g. under normal use) into the dermis.

### FIELD AND BACKGROUND OF THE INVENTION

The present invention, in some embodiments thereof, relates to a device and method for stimulating skin and, more particularly, but not exclusively, to a device and method for directly stimulating the skin below the surface of the scalp to promote hair growth.

### SUMMARY

Apparatus for treating the scalp comprises a plurality of ion-releasing electrode protrusions configured so that when first and second of the protrusions are simultaneously in contact with human skin, at least partially-ionic current flows between the first and second electrode protrusions via the skin so as to deposit ions, released from the first and/or second electrode protrusions, on the skin.

Apparatus for treating the scalp comprises a plurality of ion-releasing electrode protrusions configured so that when first and second of the protrusions are simultaneously in contact with human skin, at least partially-ionic current flows between the first and second electrode protrusions via the skin so as to deposit on the skin, a ion and a counter-ion thereof, the ion and counter-ion being released from the first and/or second electrode protrusions.

In some embodiments, the plurality comprises at least 2 or at least at least 5 or at least 10 or at least 20 or at least 30 or at least 50 or at least 75 or at least 100 or at least 150 or at least 200 or at least 300 protrusions.

In some embodiments each electrode of the plurality is respectively associated with a respective counter-electrode of the electrode plurality, optionally a lateral displacement between the electrode and its respective counter-electrode being at most 1 cm or at most 7.5 mm or at most 5 mm, to define a respective electrode-pair such that when both electrodes of the respective electrode-pair are simultaneously in contact with human skin, at least partially-ionic current flows between the electrode protrusions of the respective electrode-pair via the skin so as to deposit ions, released from any one or both electrodes of the respective electrode-pair on the skin.

In some embodiments each electrode of the plurality is respectively associated with a respective counter-electrode of the electrode plurality, optionally a lateral displacement between the electrode and its respective counter-electrode being at most 1 cm or at most 7.5 mm or at most 5 mm, to define a respective electrode-pair such that when both electrodes of the respective electrode-pair are simultaneously in contact with human skin, at least partially-ionic current flows between the electrode protrusions of the respective electrode-pair via the skin so as to deposit at least two counter-ions, released from any one or both electrodes of the respective electrode-pair on the skin.

In some embodiments the plurality is operatively coupled to mechanical actuator(s) configured to repeatedly bring pairs of the electrode into and out of contact with a surface of skin so as to form ion-deposition islands thereon.

In some embodiments each electrode of the protrusion extends from a base-surface selected from the group consisting of (i) a surface of a wheel or cylindrical roller or spherical roller or disc; (ii) a rigid flat surface; and (iii) a conformable surface that is—flat in at least one configuration.

In some embodiments, the roller is a cylindrical roller having a limited roll-range, for example, at most 270 degrees or at most 180 degrees or at most 135 degrees or at most 90 degrees or at most 60 degrees or at most 45 degrees.

In some embodiments a separation distance between the first and second protrusions is at most 1 cm or at most 5 mm.

A scalp-brush apparatus comprises a protrusion-base surface having at least one configuration where the protrusion-base surface is substantially flat; a plurality of ion-releasing electrode protrusions extending from the protrusion-base surface such that for at least one configuration of the protrusion-base surface: i. the ion-releasing electrode-protrusions are generally parallel to each other to pass through a common plane above the protrusion-base surface; and ii. for a patch-set of at least A non-overlapping square patches within the common-plane, each square-patch having an area of B mm2, respective first and second ion-releasing electrode-protrusions respectively pass through each square patch of the patch-set such that, for each patch of the patch-set, when the respective first and second ion-releasing electrode-protrusions are simultaneously in contact with human skin, an at least partially-ionic current flows between the respective first and second electrode-protrusions to deposit, onto the skin, ions that are respectively released from the respective first and/or second electrode-protrusions, wherein a value of A is selected from the group consisting of 3, 5, 7, 10, 12, 15, 20, 30, 50, 75, 100 and a value of B is selected from the group consisting of 10, 20, 30, 40, 50, 60, 70, 80, 100, 150, 200, 250, and 300.

A scalp-brush apparatus comprises a protrusion-base surface having at least one configuration where the protrusion-base surface is substantially flat; a plurality of ion-releasing electrode protrusions extending from the protrusion-base surface such that for at least one configuration of the protrusion-base surface: i. the ion-releasing electrode-protrusions are generally parallel to each other to pass through a common plane above the protrusion-base surface; and ii. for a patch-set of at least A non-overlapping square patches within the common-plane, each square-patch having an area of B mm2, respective first and second ion-releasing electrode-protrusions respectively pass through each square patch of the patch-set such that, for each patch of the patch-set, when the respective first and second ion-releasing electrode-protrusions are simultaneously in contact with human skin, an at least partially-ionic current flows between the respective first and second electrode-protrusions to deposit, onto the skin, a ion and a counter-ion thereof, the ion and counter-ion being released from the respective first and/or second electrode protrusions

wherein a value of A is selected from the group consisting of 3, 5, 7, 10, 12, 15, 20, 30, 50, 75, 100 and a value of B is selected from the group consisting of 10, 20, 30, 40, 50, 60, 70, 80, 100, 150, 200, 250, and 300.

In some embodiments, the set of patches cover at least a rectangular region of the common plane having a length of X mm and a width of Y mm, wherein (I) a value of X of is at least 10 mm, or at least 20 mm or at least 30 mm or at least 40 mm and/or at most 100 mm or at most 75 mm or at most 50 mm or at most 40 mm or at most 30 mm or at most 20 mm and (ii) a value of Y is at least 30 mm or at least 40 mm or at least 50 mm or at least 60 mm or at least 70 mm or at least 80 mm or at least 90 mm or at 100 mm and/or at most 150 mm or at most 120 mm or at most 100 mm or at most 80 mm or at most 60 mm or at most 50 mm or at most 40 mm.

In some embodiments, protrusion-base surface has a first configuration where the protrusion-base surface is flat so that electrode-protrusions extending therefrom are parallel to each other and a second configuration where the protrusion-base is concave at least in one direction so that electrode-protrusions extending therefrom converge, an angle of convergence being at least 15 degrees. Alternatively, the base of the brush from which the protrusions extend may be concave in contrast to FIG. 14A to obtain a distal-end shape like in FIG. 15C

A scalp-treatment apparatus comprises a round roller (e.g. cylindrical or spherical) or

a plurality of ion-releasing electrode protrusions extending from a surface of the round roller to pass through a round common-surface above the surface of the round roller such that, for a patch-set of at least A non-overlapping patches within the round common-surface, each patch being square within the round common-surface relative to the curvilinear coordinates defined by the common surface, each curvilinear-coordinate-relative-square-patch having an area of B mm2, respective first and second ion-releasing electrode-protrusions respectively pass through each curvilinear-coordinate-relative-square patch of the patch-set such that, for each patch of the patch-set, when the respective first and second ion-releasing electrode-protrusions are simultaneously in contact with human skin, an at least partially-ionic current flows between the respective first and second electrode-protrusions to deposit, onto the skin, ions that are respectively released from the respective first and/or second electrode-protrusions, a value of A is selected from the group consisting of 3, 5, 7, 10, 12, 15, 20, 30, 50, 75, 100 and a value of B is selected from the group consisting of 10, 20, 30, 40, 50, 60, 70, 80, 100, 150, 200, 250, and 300.

A scalp-treatment apparatus comprises a round roller (e.g. cylindrical or spherical) or a plurality of ion-releasing electrode protrusions extending from a surface of the round roller to pass through a round common-surface above the surface of the round roller such that, for a patch-set of at least A non-overlapping patches within the round common-surface, each patch being square within the round common-surface relative to the curvilinear coordinates defined by the common surface, each curvilinear-coordinate-relative-square-patch having an area of B mm2, respective first and second ion-releasing electrode-protrusions respectively pass through each curvilinear-coordinate-relative-square patch of the patch-set such that, for each patch of the patch-set, when the respective first and second ion-releasing electrode-protrusions are simultaneously in contact with human skin, an at least partially-ionic current flows between the respective first and second electrode-protrusions to deposit, onto the skin, a ion and a counter-ion thereof, the ion and counter-ion being released from the respective first and/or second electrode protrusions wherein, (I) a value of A is selected from the group consisting of 3, 5, 7, 10, 12, 15, 20, 30, 50, 75, 100 and (II) a value of B is selected from the group consisting of 10, 20, 30, 40, 50, 60, 70, 80, 100, 150, 200, 250, and 300.

In some embodiments wherein the roller is continuous along its central axis.

In some embodiments wherein the roller comprises a disc-array of at least two or at least 3 or at least 4 or at least 5 or at least 10 thin co-axial discs spaced along a roller central axis, the electrode-protrusions being disposed around a circumference of each of the discs and radially protruding therefrom.

In some embodiments, a thickness of the each thin-disc is 0.75 mm or at most 0.5 mm or at most 0.25 mm or at most 0.1 mm.

In some embodiments wherein all discs of the disc-array rotate in-tandem with each other.

In some embodiments wherein for each pair of neighboring discs, inter-disc distance therebetween along the roller central axis (i) at least 2 mm and/or (ii) at most 1 cm or at most 8 cm at most 6 mm and/or (iii) at least 5 times or at least 10 times or at least 20 time a thickness of a thickest disc of the disc-array.

In some embodiments wherein each disc has a diameter of at least 10 mm, or at least 20 mm, or at least 30 mm, or at least 40 mm, or at least 50 mm, or at least 60 mm, or at least 70 mm.

In some embodiments wherein configured so that for at least one pair of neighboring discs, an annular portion of an inter-disc region therebetween is substantially void, wherein (i) a length of the annular portion is at 20% or at least 30% or at least 40% or at least 50% an inter-disc distance between the neighboring discs; and (ii) an outer diameter of the annular portion is at least that of the neighboring discs; and (iii) an inner diameter of the annular portion is at most 5 mm or at most 10 mm less than that the of the neighboring discs.

A scalp-treatment apparatus comprises a disc-array of at least two or at least 3 or at least 4 or at least 5 or at least 10 co-axial discs (e.g. thin discs) spaced along a roller central axis; ion-releasing electrode-protrusions being disposed around a circumference of each of the discs and radially protruding therefrom such that when first and second of the protrusions are simultaneously in contact with human skin, at least partially-ionic current flows between the first and second electrode protrusions via the skin so as to deposit ions, released from the first and/or second electrode protrusions, on the skin.

A scalp-treatment apparatus comprises a disc-array of at least two or at least 3 or at least 4 or at least 5 or at least 10 co-axial discs (e.g. thin discs) spaced along a roller central axis;

b. ion-releasing electrode-protrusions being disposed around a circumference of each of the discs and radially protruding therefrom such that when first and second of the protrusions are simultaneously in contact with human skin, at least partially-ionic current flows between the first and second electrode protrusions via the skin so as to deposit on the skin, a ion and a counter-ion thereof, the ion and counter-ion being released from the first and/or second electrode protrusions.

In some embodiments wherein the roller is continuous along its central axis.

In some embodiments wherein the roller comprises a disc-array of at least two or at least 3 or at least 4 or at least 5 or at least 10 thin co-axial discs spaced along a roller central axis, the electrode-protrusions being disposed around a circumference of each of the discs and radially protruding therefrom.

In some embodiments, a thickness of the each thin-disc is 0.75 mm or at most 0.5 mm or at most 0.25 mm or at most 0.1 mm

In some embodiments wherein wherein all discs of the disc-array rotate in-tandem with each other.

In some embodiments wherein for each pair of neighboring discs, inter-disc distance there between along the roller central axisis at least 2 mm and/or at most 1 cm or at most 8 cm at most 6 mm and/or at least 5 times or at least 10 times or at least 20 time a thickness of a thickest disc of the disc-array.

In some embodiments wherein each disc has a diameter of at least 10 mm, or at least 20 mm, or at least 30 mm, or at least 40 mm, or at least 50 mm, or at least 60 mm, or at least 70 mm

In some embodiments wherein configured so that for at least one pair of neighboring discs, an annular portion of an inter-disc region there between is substantially void, wherein a length of the annular portion is at 20% or at least 30% or at least 40% or at least 50% an inter-disc distance between the neighboring discs; and an outer diameter of the annular portion is at least that of the neighboring discs; and an inner diameter of the annular portion is at most 5 mm or at most 10 mm less than that the of the neighboring discs.

In some embodiments wherein there is a round-roller (e.g. cylinder but not only) comprising the cylindrical roller and/or wherein the one or more discs along the common rotation axes so that outer diameters thereof substantially lie along a common geometrical cylinder, thereby defining a cylindrical roller, wherein the cylindrical roller has a limited roll-range, for example, at most 270 degrees or at most 180 degrees or at most 135 degrees or at most 90 degrees or at most 60 degrees or at most 45 degrees.

In some embodiments wherein at least sensor configured to sense at least one parameter related to operation of the apparatus and/or to a status of skin treated by the apparatus; and (ii) at least one response-element configured to generate a response, responsively to the results of the sensing.

In some embodiments, at least one sensor(s) is selected from the group consisting of an ion-deposition rate sensor configured to sense a rate of deposition of ions on the skin by the at least partially-ionic current; a force or pressure sensor configured to sense an amount of force or pressure between the electrode-protrusion(s) and skin; skin-color sensor, a current sensor configured to sense a magnitude of current via the skin via electrode-protrusions; a trapped or tangled hair sensor configured to sense a presence or absence or amount of hair trapped within or entangled to the roller (e.g. mechanical and/or optical); a skin wetness sensor; a skin temperature sensor (e.g. based on IR); and a scalp thickness sensor (e.g. based on ultrasound). IX. ROLL COUNTER; X.

### Accelerometer

In some embodiments wherein at least one response-element is selected from the group consisting of a vibration controller configured to control at least one of an amplitude, frequency, direction, relative-amplitude of mechanical vibrations of the electrode-protrusion(s); an alert-signal generator configured to generate an alert signal (e.g. visual and/or audio and/or tactile); a session-duration regulator configured to regulate a duration of a treatment session (e.g. by signaling a ‘session alert’ alert or by shutting off the vibrations and/or the light and/or the electrical current driving ion deposition); an inter-protrusion voltage-regulator configured to regulate a voltage between electrode protrusions (e.g to increase a voltage by a factor of at least 2 or at least 5 or at least 10; e.g. to generate a series of pulses); a roller-resistance or disc-rolling-resistance controller (e.g. mechanical and/or electrical) configured to regulate a degree of resistance to rolling of the disc and/or roller (e.g. to increase the resistance if the ‘effectiveness of treatment’—e.g. current between electrodes - - - is too low; a depth-penetration controller configured to regulate a depth to which tips of the electrode-protrusions penetrate the skin (e.g. by regulating a length of the mini-needle or a location of the stopper); a base-surface shape-regulator configured to regulate an extent of a deviation from flatness of the generally-flat base-surface from which the protrusions extend.

In some embodiments wherein the response element responds to the results of the sensing in accordance with a number of previous sessions that the device has been used.

In some embodiments the plurality of electrode protrusions configured, when at least two of the protrusions are simultaneously in contact with human skin, at least partially-ionic electric current flows between first and second protrusions via the skin, wherein the protrusions are disposed around the circumference of a wheel or roller having only partial rotational freedom.

In some embodiments wherein at least one of, or at least a plurality of, or at least a majority of the electrode protrusions are blunt at distal ends thereof.

In some embodiments wherein at least one of, or at least a plurality of, or at least a majority of the electrode protrusions are sharp at distal ends thereof.

In some embodiments wherein at least one, or at least a plurality of, or at least a majority of the electrode-protrusion comprises an electrode-protrusion main body (for example, characterized by a greater-thickness of at most 2 mm and/or a length of at least 0.2 mm) the main-body being blunt at its distal end and/or the main-body having a blunt distal-facing surface; and one or more sharp mini-needle(s) extending from the blunt distal end or the blunt distal-facing surface of the main body, the mini-needle being sharp at a distal surface thereof.

In some embodiments, a thickness of the sharp mini-needle is at most 100 microns and/or a length the sharp mini-needles is at least 10 microns or at least 20 microns and/or at most thereof being between 10 and 150 microns.

In some embodiments wherein at a location distanced 50 microns from a tip of the electrode protrusion, a cross-section of the electrode-protrusion is at least 0.001 mm̂2, or at least 0.005 mm̂2, or at least 0.01 mm̂2, or at least 0.02 mm̂2, or at least 0.05 mm̂2

In some embodiments wherein when a tip of the electrode-scalp is brought into contact with a healthy human scalp, an electrode-scalp contact area for each electrode-scalp contact event is at most 10 mm2

In some embodiments wherein configured to regulate a maximum skin-penetration-depth of electrode-protrusions to at most 100 microns or at most 75 microns or at most 50 microns or at most 20 microns when a tip of the electrode-protrusion is pressed against a healthy human scalp at a pressure of 0.1 to 5 MPa.

In some embodiments wherein at least one of, or at least a plurality of, or at least a majority of the electrode protrusions are flexible, for example, to provide a variation in a base-tip distance of at least 1 mm or at least 2 mm or least 3 mm or at least 5 mm or at least 7 mm or at least 10 mm and/or at least 10% of (or at least 25% of, or at least 50% of) a length of the electrode-protrusion. In some embodiments wherein an electrical power source configured to at least partially drive the at least partially-ionic current between electrode-protrusions via the skin to at least partially drive the ion deposition thereon.

In some embodiments, the electrical power source produces time varying current between the electrodes, for example, alternating current, for example, at a frequency of at least 0.1 Hz and/or at most 10 Hz.

In some embodiments wherein each of the electrode-protrusions is laterally distanced from its nearest neighbor by at most 1 cm or at most 5 mm

In some embodiments wherein an onboard source(s) of mechanical vibration to vibrate each of the electrode-protrusions in at least one or in both lateral-directions and/or along a lateral direction defined by the electrode-protrusion.

In some embodiments wherein a light source, for example, configured to emit primarily light having a wavelength of at least about 620 nm and at most about 680 nm, for example, a LED or laser or source of coherent light.

In some embodiments wherein the ion-releasing electrodes are configured as hybrid light guide:ion-releasing electrodes so that light received from the light source longitudinally travels within the hybrid light guide:ion-releasing electrodes, for example, so that the light exits from the hybrid light guide:ion-releasing electrodes along the longitudinal direction of the hybrid light guide:ion-releasing electrode - - - for example, constructed of a transparent polymer either electrically conducting or coated by an electrically-conducting substance—e.g. comprising metal ions.

In some embodiments wherein employing the apparatus to deposit metal ions on the scalp and/or to provide a massage thereto and/or to illuminate the scalp.

In some embodiments wherein employing the apparatus to deposit metal ions on the scalp and/or to provide a massage thereto and/or to illuminate the scalp.

In some embodiments, for treating or preventing a hair-condition of a user, the user's scalp dividable into a scalp-patch-set of n mm x n mm non-overlapping square scalp patches, n being a positive number having a value of at most 5, the apparatus comprises means for subjecting the user's scalp to at least q distinct electrode-scalp contact events within a time-interval of at most one minute, the time interval being dividable into m non-overlapping equal-duration sub-intervals covering the time-interval, m being a positive integer having a value of at least 5, q being a positive integer having a value of at least 200, the method performed such that for at least a majority of the electrode-scalp contact events, no electrode of the event enters into the dermis; a duration of each electrode-scalp contact event is at most 100 milliseconds; an electrode-scalp contact area for each electrode-scalp contact event is at most 10 mm2; for each electrode-scalp contact event, an electrical current flows between the electrode and the scalp so as to deposit electrode-released ions of a first metal or of a second metal on the scalp, thereby forming a respective metal-ion-deposition island on the user's scalp; for each of the m non-overlapping equal-duration sub-intervals, at least p electrode-scalp contact events occur, p being a positive integer having a value of at least 1; at least 5% of the electrode-scalp contact events are first-metal-depositing and at least 5% of the electrode-scalp contact events are second-metal-depositing; and at least one first-metal-deposition-island and at least one second-metal-deposition-island are both respectively and distinctly formed on each n mm x n mm scalp scalp-patch selected from a 10-member scalp-patch sub-set of the scalp-patch set.

In some embodiments, treating or preventing a hair-condition of a user, the user's scalp dividable into a scalp-patch-set of n mm x n mm non-overlapping square scalp patches, n being a positive number having a value of at most 5, the method comprises subjecting the user's scalp to at least q distinct electrode-scalp contact events within a time-interval of at most one minute, the time interval being dividable into m non-overlapping equal-duration sub-intervals covering the time-interval, m being a positive integer having a value of at least 5, q being a positive integer having a value of at least 200, the method performed such that for at least a majority of the electrode-scalp contact events, no electrode of the event enters into the dermis; a duration of each electrode-scalp contact event is at most 100 milliseconds; an electrode-scalp contact area for each electrode-scalp contact event is at most 10 mm2; for each electrode-scalp contact event, an electrical current flows between the electrode and the scalp so as to deposit electrode-released ions of a first metal or of a second metal on the scalp, thereby forming a respective metal-ion-deposition island on the user's scalp;

v. for each of the m non-overlapping equal-duration sub-intervals, at least p electrode-scalp contact events occur, p being a positive integer having a value of at least 1; at least 5% of the electrode-scalp contact events are first-metal-depositing and at least 5% of the electrode-scalp contact events are second-metal-depositing; and at least one first-metal-deposition-island and at least one second-metal-deposition-island are both respectively and distinctly formed on each n mm x n mm scalp scalp-patch selected from a 10-member scalp-patch sub-set of the scalp-patch set.
In some embodiments wherein during at least some of the electrode-scalp contact events, externally-generated electrical current is respectively forced between the electrode and the scalp so as to respectively deposit or increase a deposition-rate of electrode-released ions of the first or second metal onto the scalp.

In some embodiments wherein a value of q is at least 1000.

In some embodiments wherein a value of p is at least 5.

In some embodiments wherein a value of m is at least 10.

In some embodiments wherein a value of p is at least 5.

In some embodiments wherein for at least 75% of the electrode-scalp contact events, no electrode enters into the dermis.

In some embodiments wherein at least 20% of the events are first-metal-depositing.

In some embodiments wherein at least 20% of the events are second-metal-depositing.

In some embodiments wherein at least four metal-deposition-islands are respectively and distinctly formed on each n mm x n mm scalp-patch selected from a 10-member scalp-patch sub-set of the scalp-patch set, the four metal-deposition islands comprising at least two first-metal-depositing islands and at least two second-metal-depositing-islands.

In some embodiments wherein a duration of each electrode contact event is at most 50 milliseconds.

In some embodiments wherein a duration of each electrode contact event is at most 25 milliseconds.

In some embodiments wherein an electrode-scalp contact area for each electrode-scalp contact event is at most 5 mm2

In some embodiments wherein during each of a majority of the electrode-scalp contact events, the scalp is respectively subjected to an electrode-applied pressure of at least 0.5 mega-Pascals.

In some embodiments wherein during each of at least 75% of the electrode-scalp contact events, the scalp is respectively subjected to an electrode-applied pressure of at least 0.5 mega-Pascals.

In some embodiments wherein during each of a majority of the electrode-scalp contact events, the scalp is respectively subjected to an electrode-applied pressure of at least 1 mega-Pascal.

In some embodiments wherein during each of at least 75% of the electrode-scalp contact events, the scalp is respectively subjected to an electrode-applied pressure of at least 1 mega-Pascal.

In some embodiments wherein a value of q is at least 250, and wherein for each of the electrode-scalp contact events, at least some of the released metal-ions deposited on the scalp are provided from an electrode interior of the electrode and/or from an electrode metal-coating that is integrally formed with the electrode.

In some embodiments wherein during a majority of the electrode-scalp contact events, externally-generated electrical current is forced between the electrode and the scalp so as to deposit or increase a deposition-rate of electrode-released ions of the first or second metal onto the scalp.

Apparatus for treating the scalp comprising:

a plurality of ion-releasing electrode protrusions configured so that when first and second of the protrusions are simultaneously in contact with human skin, at least partially-ionic current flows between the first and second electrode protrusions via the skin so as to deposit ions, released from the first and/or second electrode protrusions, on the skin.

Apparatus for treating the scalp comprising:

a plurality of ion-releasing electrode protrusions configured so that when first and second of the protrusions are simultaneously in contact with human skin, at least partially-ionic current flows between the first and second electrode protrusions via the skin so as to deposit (e.g. sequentially) on the skin, a ion and a counter-ion thereof (for example, to cycle back and forth between a first-mode where the ion is deposited without the counter ion and a second mode where the counter ion is deposited without simultaneously depositing the ion), the ion and counter-ion being released from the first and/or second electrode protrusions. In some embodiments, a separation distance between the first and second protrusions is at most 1 cm or at most 5 mm

Embodiments of the invention relate to a device and method whereby the scalp is rapidly and repeatedly touched by ion-releasing electrodes. During each ‘ion-depositing electrode-scalp contact event’ an electrode (e.g. through which externally generated electrical current flows) is very briefly brought into and out of contact with the scalp—e.g. in contact with the scalp for at most 100 milliseconds. During each brief contact event, the electrode is briefly brought into and out of contact with the scalp so as to deposit metal on the scalp to form a small (e.g. at most 15 mm2 in area) metal-deposition island on the scalp. In some embodiments, each brief contact event is effective to apply a significant amount of highly-localized pressure. e.g at least 0.5 megapascals [MPa] localized over a contact area of at least 0.1 mm2 and at most 10 mm2 The rapid application of non-wounding but significant pressure subjects the scalp to a ‘micromassage.’

The method is performed so that: (i) a large number of such electrode-events are sequentially performed within a relatively short period of time; (ii) at least two types of metal-deposition islands are formed on the scalp (e.g. a first type comprising zinc and a second type comprising copper); and (iii) both types of metal-deposition islands are distributed over a significant portion of the scalp. As discussed below, it is possible to quantify the extent of distribution of metal-islands on the scalp and the proximity of first and second types of metal islands (e.g. ‘cathode-islands’ and ‘anode-islands’), in terms of ‘scalp patches.’

Not wishing to be bound by theory, it is believed that the deposition of a relatively large number of very small but distinct metal-ion-deposition-islands on the user's scalp forms a significant number of ‘micro-battery-cell’ on the user's scalp when both cation islands and anion islands are distributed over a region of the scalp. It is believed that after deposition of the islands, small electrical currents may be sustained between the distinct deposition islands (e.g. due to proximity of distinct cathode-islands and anode-islands) along the user's scalp for some period of time (e.g. at least hours). It is believed that the combination of the time-sustained electrical stimulation together with the mild trauma of the micro-massage obviates the need to employ wounding-based techniques to stimulate the scalp.

Although skin-wounding stimulates cell-growth in the skin (and possibly hair-growth) by inducing a biological ‘wound-healing’ process, certain users may consider wounding devices as invasive and unpleasant to use. It is believed that the presently-disclosed ion-delivering micro-massage obviates the need for a more severe treatment regimen based on wounding, while still combating baldness.

When metallic-ions are ‘released from’ an electrode this is in contrast with pre-applying an ion-containing topical agent (e.g. an ion-containing liquid or cream or gel) to the skin and then using an electrode to drive the ions into the skin. When metallic-ions are ‘released from’, the source of the metallic ions is from the electrode itself. The released metal-ions are provided from an interior of the electrode (e.g. from a reservoir disposed within the electrode) or from actual material of the electrode (i.e. the electrode is at least partially constructed from the metal which is then released) or from an ‘integrally-formed’ coating on the electrode—i.e. the electrode is pre-coated with the metal so that the metal coating is integrally formed with the electrode and then metal of this coating is released.

By ‘releasing’ metallic ions from the electrode rather than relying on a topically-applied ion-containing flowable-fluid (e.g. liquid, cream, gel), it is possible to deliver distinct ion-deposition metal-ion deposition islands. After treatment, small electrical currents may flow between these metal-deposition islands to electrically stimulate the skin after the electrode-contacting events have ceased, thereby providing a sustained effect.

A number of techniques are disclosed herein for rapidly bringing electrode into and out of contact with the scalp. In one example, a plurality of electrode-protrusions (e.g. having a rounded tip) are disposed around a roller. As the roller is rolled over the surface of the skin, the electrodes are briefly brought into contact with and out of contact with the skin so that a large number of very brief electrode contact events are performed. A second example relates to a motorized device. In this second example, electrodes (eg. having a rounded tip) are rapidly, reciprocally and vertically brought into contact and out of contact with the scalp.

Despite the very-brief contact periods (i.e. less than 0.1 seconds or even less) between each electrode and the scalp, a therapeutically effective amount of metallic-ions may be deposited in each treatment island. Towards this end, an external electrical power source may boost a rate of ion-delivery to each treatment island, instead of relying only on a galvanic potential between electrodes of different polarity. Not wishing to be bound by theory, externally-driving ion deposition on the scalp may, once again, obviate the need for a more mechanically-aggressive wounding-based treatment where most electrode-contact events lead to penetrating of the dermis.

It is now disclosed a method of treating or preventing a hair-condition of a user, the user's scalp dividable into a scalp-patch-set of n millimeter (mm) x n millimeter (mm) non-overlapping scalp patches, where n a positive number having a value of at most 5. The method comprises subjecting the user's scalp to at least q distinct electrode-scalp contact events within a time-interval of at most one minute and dividable into m non-overlapping equal-duration sub-intervals covering the time-interval, m begin a positive integer having a value of at least 5, q being a positive integer having a value of at least 200. For the non-limiting example where m is 5, the m equal-duration sub-intervals are [0.12 seconds], [12 seconds, 24 seconds], [24 seconds, 36 seconds], [36 seconds, 48 seconds], and [48 seconds, 60 seconds]. Since every moment within the one-minute time interval i within one of the sub-intervals, the sub-intervals may be said to collectively ‘cover an entirety of the time-interval.

In some embodiments, for at least a majority of the electrode-scalp contact events, no electrode of the event enters into the dermis;

In some embodiments, a duration of each electrode scalp contact event is at most 100 milliseconds—i.e. for each electrode-scalp no more than 100 milliseconds elapses between (i) a time when the electrode is brought into contact with the scalp; and (ii) a time when the electrode is taken out of contact with the scalp.

In some embodiments, an electrode-scalp contact area for each electrode-scalp contact event is at most 10 mm².

In some embodiments, for each electrode contact event, an electrical current flows between the electrode and the scalp so as to deposit electrode-released ions of a first metal or of a second metal on the scalp, thereby forming a respective metal-ion-deposition island on the user's scalp. Thus, each contact event deposits either a first metal (e.g. zinc) and a second metal (e.g. copper) but not both.—other metals other than the first and second metal may additionally be deposited along with the first or the second metal.

In some embodiments, for each of the non-overlapping equal-duration sub-intervals, at least p electrode-scalp contact events occur, p being a positive integer having a value of at least 1.

In some embodiments, at least 5% of the events are first-metal-depositing and at least 5% of the events are second-metal-depositing.

In some embodiments, at least one first-metal-deposition-island and at least one second-metal-deposition-island are both respectively and distinctly formed on each n mm x n mm scalp-patch selected from a 10-member scalp-patch sub-set of the scalp-patch set.

In some embodiments, the islands may be ‘distinct’ from each other for some the islands may form a bridge′ between the formerly-′distinct′ islands. This does not detract from the fact that for at least some period of time, the islands were ‘distinct’ from each other.

In some embodiments, during at least some of the electrode-scalp contact events, externally-generated electrical current (i.e. as opposed to galvanic current) is forced between the electrode and the scalp (for example, between two different electrodes that are simultaneously in contact with the scalp where due to an externally-maintained electric potential difference between the electrodes, electrical current flows therebetween via the scalp) so as to deposit or increase a deposition-rate of electrode-released ions of the first or second metal onto the scalp. Some galvanic current may be present, but the externally-generated electrical current may boost a rate of metal-ion-deposition.

A cosmetic method of treating or preventing a hair-condition of a user comprising: subjecting the user's scalp to at least 200 distinct electrode-scalp contact events during a time-interval of at most one minute and dividable into 5 non-overlapping equal-duration sub-intervals covering the time-interval, method performed such that i. for at least a majority of the electrode-scalp contact events, no electrode

of the event enters into the dermis; ii. a duration of each electrode contact event is at most 100 milliseconds; and iii. for each electrode contact event, an electrical current flows between the electrode and the scalp so as to deposit electrode-released ions of a first metal or of a second metal on the scalp, thereby forming a respective metal-ion-deposition island on the user's scalp.

### BRIEF DESCRIPTION OF THE DRAWINGS

Some embodiments of the invention are herein described, by way of example only, with reference to the accompanying drawings and/or images. With specific reference now to the drawings and/or images in detail, it is stressed that the particulars shown are by way of example and for purposes of illustrative discussion of embodiments of the invention. In this regard, the description taken with the drawings and/or images makes apparent to those skilled in the art how embodiments of the invention may be practiced.

In the drawings:

FIGS. 1-5 relate to a device or portion(s) thereof for depositing metal ions on the scalp, for example, to treat a hair-condition such as baldness.

FIGS. 6 and 9 illustrate patterns of metal-ion-deposition on the scalp.

FIG. 7 illustrates a timeline showing where electrodes are brought into contact and out of contact with the scalp.

FIG. 8 illustrates a contact-event between electrodes and skin (e.g. of the scalp) wherein the electrodes do not penetrate into the dermis and ions are deposited on the skin (e.g. of the scalp).

FIGS. 10A-10D, 11A-11I, 12A-12D, 13A-13D, 14A-14C, 15A-15D, 16A-16D, 17, 18A-18B, 19-26 relate to additional embodiments of treating the scalp.

FIGS. 27-28 describe some experimental results.

### DESCRIPTION OF SPECIFIC EMBODIMENTS OF THE INVENTION

The invention is herein described, by way of example only, with reference to the accompanying drawings. With specific reference now to the drawings in detail, it is stressed that the particulars shown are by way of example and for purposes of illustrative discussion of the preferred embodiments of the exemplary system only and are presented in the cause of providing what is believed to be a useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how several forms of the invention may be embodied in practice and how to make and use the embodiments.

For brevity, some explicit combinations of various features are not explicitly illustrated in the figures and/or described. It is now disclosed that any combination of the method or device features disclosed herein can be combined in any manner—including any combination of features—and any combination of features can be included in any embodiment and/or omitted from any embodiments.

### DEFINITIONS

For convenience, in the context of the description herein, various terms are presented here. To the extent that definitions are provided, explicitly or implicitly, here or elsewhere in this application, such definitions are understood to be consistent with the usage of the defined terms by those of skill in the pertinent art(s). Furthermore, such definitions are to be construed in the broadest possible sense consistent with such usage.

In the present disclosure ‘electrical circuitry’ or ‘electronic circuitry’ is intended broadly to describe any combination of hardware, software and/or firmware.

Electronic circuitry may include may include any executable code module (i.e. stored on a computer-readable medium) and/or firmware and/or hardware element(s) including but not limited to field programmable logic array (FPLA) element(s), hard-wired logic element(s), field programmable gate array (FPGA) element(s), and application-specific integrated circuit (ASIC) element(s). Any instruction set architecture may be used including but not limited to reduced instruction set computer (RISC) architecture and/or complex instruction set computer (CISC) architecture. Electronic circuitry may be located in a single location or distributed among a plurality of locations where various circuitry elements may be in wired or wireless electronic communication with each other.

When metallic-ions are ‘released from’ an electrode this is in contrast with pre-applying an ion-containing topical agent (e.g. an ion-containing liquid or cream or gel) to the skin and then using an electrode to drive the ions into the skin. When metallic-ions are ‘released from’, the source of the metallic ions is from the electrode itself. The released metal-ions are provided from an interior of the electrode (e.g. from a reservoir disposed within the electrode) or from actual material of the electrode (i.e. the electrode is at least partially constructed from the metal which is then released) or from an ‘integrally-formed’ coating on the electrode—i.e. the electrode is pre-coated with the metal so that the metal coating is integrally formed with the electrode and then metal of this coating is released.

By ‘releasing’ metallic ions from the electrode rather than relying on a topically-applied ion-containing flowable-fluid (e.g. liquid, cream, gel), it is possible to deliver distinct ion-deposition metal-ion deposition islands. After treatment, small electrical currents may flow between these metal-deposition islands to electrically stimulate the skin after the electrode-contacting events have ceased, thereby providing a sustained effect.

A ‘counter-ion’ is an ion with a different electrochemical potential relatively to the skin.

Typically, an ion is a ‘metal ion.’

A ‘CYLINDRICAL ROLLER’ is either continuous—full cylinder—OR a series of discs along a common central axis (straight or conformable) where the circumferences of the discs are substantially disposed along a common ‘geometrical-construct’ cylinder’]

A ‘thin disc’ having a thickness of at most 1 mm or at most 0.75 mm or at most 0.5 mm or at most 0.25 mm or at most 0.1 mm and/or a diameter of at least 10 mm, or at least 30 mm or at least 40 mm or at least 50 mm or at least 60 mm or at least 70 mm

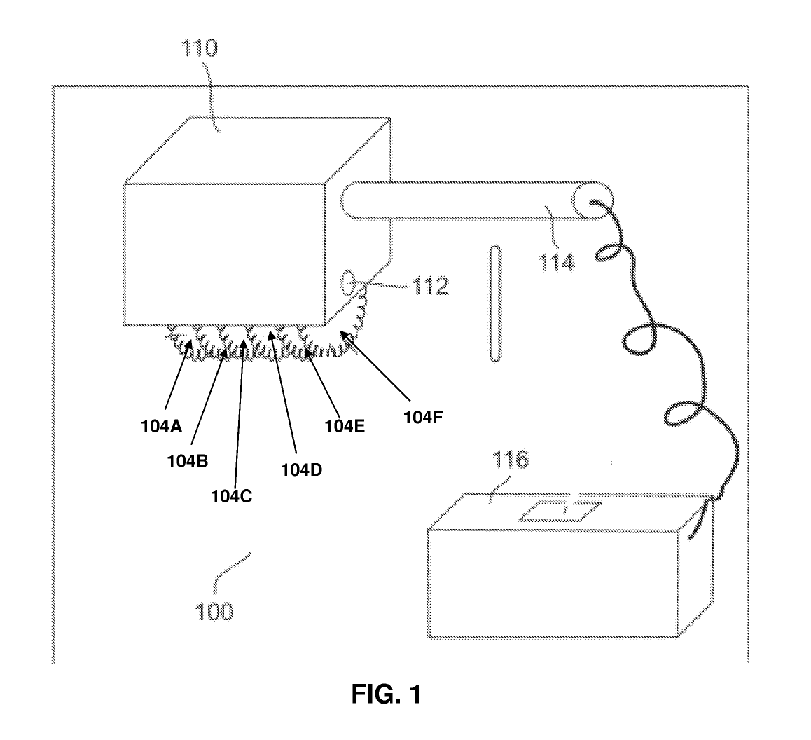

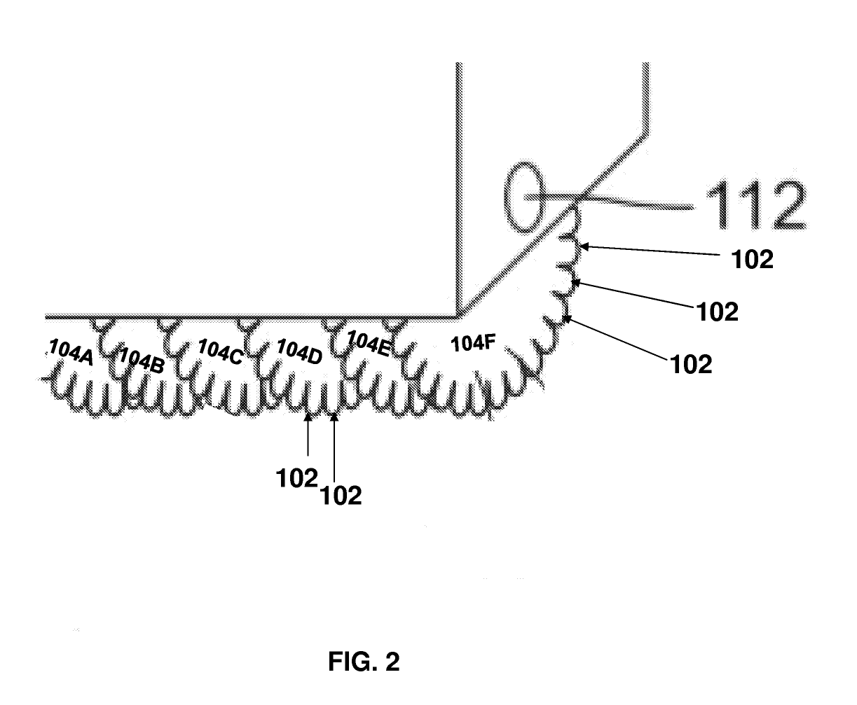

FIG. 1 is an illustration of an exemplary device 100 for promoting hair growth, in accordance with an exemplary embodiment of the invention. FIG. 2 is a close-up of a portion of the device of FIG. 1. As illustrated in FIGS. 1-2, protrusion-electrodes 102 are arranged along the circumference of at least one disc 104, for example, 2, 4, 6, 8, or other smaller, intermediate or larger numbers of discs 104 are used. In FIG. 1, six discs labeled as 104A-104F, are illustrated. The diameter of discs 104 is, for example, about 2 cm, about 4 cm, about 6 cm, or other smaller, intermediate or larger diameters are used. The thickness of discs and/or electrodes is, for example, about 0.05 mm, about 0.1 mm, about 0.15 mm, or other smaller, intermediate or larger thickness are used.

In some embodiments, the protrusion-electrode as ‘ion-releasing’ as discussed below. Nevertheless, this is not a limitation—in fact, any feature or combination or feature(s) or embodiment referring to or requiring ‘ion-releasing electrode’ may, in other embodiments, also refer to an electrode that is not ion-releasing in any context in the present document.

In an exemplary embodiment of the invention, electrodes 102 and/or discs 104 are arranged to allow existing hair on the scalp to be displaced (e.g., brushed) away from the electrodes during use. Optionally, discs 104 are arranged parallel to one another, to allow hair to be brushed between the discs. Discs 104 are located about 1 mm apart, 3 mm apart, about 5 mm apart, or other smaller, intermediate or larger distances are used.

The shapes of the protrusions 102 are non-limiting—other examples (which may be used in any embodiment including but not limited to roller-relating embodiments, scalp-brush related embodiments)

In an exemplary embodiment of the invention, electrodes 102 are coated by at least one metal. Alternatively, electrodes 102 are made from the metal.

In one non-limiting example related to FIGS. 1-2, a first set of discs (e.g. discs 104A, 104C, and 104E) are coated with a cation (e.g. copper) while a second set of discs (e.g. 104B, 104D and 104F) are coated with an anion (e.g. zinc). In this situation, (i) metal deposition ions comprising the cation are formed contact of electrodes by discs of the first set and (ii) metal deposition ions comprising the anion are formed contact of electrodes by discs of the second set.

As will be discussed below, the alternating cation/anion disc pattern described in the previous paragraph may be useful for ensuring that, after treatment, metal-ion-deposition islands comprising the cation are relatively proximate on the scalp to metal-ion-deposition islands comprising the anion. This may be useful for depositing miniature half-batteries on the user's scalp so that small currents between the deposition islands are sustained after treatment.

For the present disclosure, when a ‘metal-ion-deposition island’ is formed there is a localized region of scalp wherein for at least one metal, the ion is deposited within the ‘deposition island’

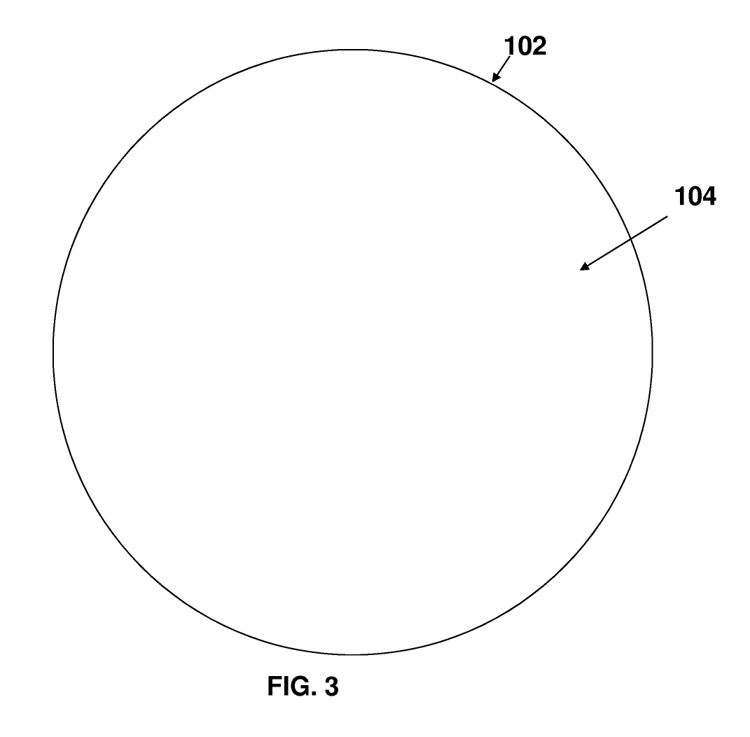

FIG. 3 illustrates an exemplary disc including a plurality of distinct protruding electrodes 102 disposed uniformly around the disc 104. The ‘uniform distribution feature’ is not intended as a limitation.

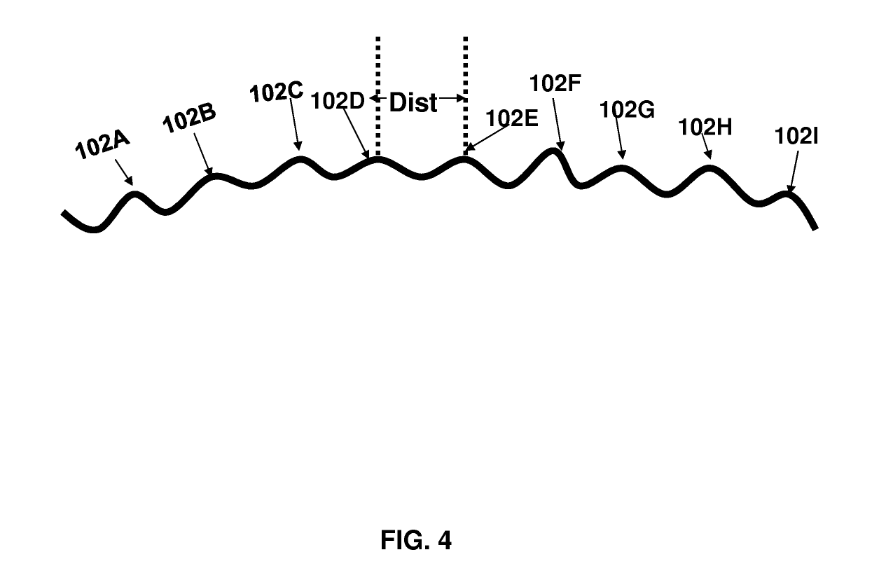

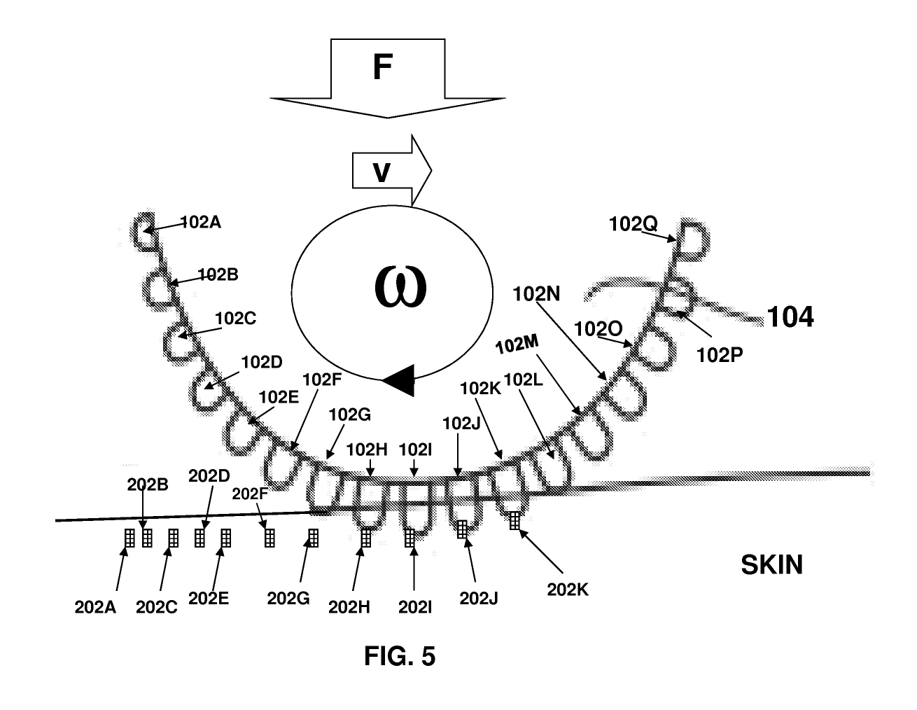

FIG. 4 is a close-up illustration of 10 electrodes 102A-102D of a disc illustrating an inter-electrode distance Dist. FIG. 5 illustrates application of a plurality of distinct metal-ion-deposition ions on the surface of the scalp (i.e. the skin thereof) by rolling, without slipping, a disc over the surface of the scalp. In the non-limiting example of FIG. 5, a center of mass of the roller moves linearly and horizontally from left-to-right (i.e. defining a direction of disc velocity v) as a result of counterclockwise rotation. A downward force F is applied in a direction normal to the scalp, or a local surface thereof. As will be discussed below, in some embodiments, when the downward force is localized along a contact-area of each the electrode, a pressure of at least 0.5 mega-Pascals per electrode may be applied to the scalp.

In the example of FIG. 5, whenever an electrode is brought into contact with the skin, the electrode releases metal ions (i.e. either from an interior of the electrode or from a metal-coating that is integrally formed with the electrode). In the example of FIG. 5, electrodes 102A-102K respectively form metal deposition islands 202A-202K. As illustrated in FIG. 5, in a direction parallel to vector v (representing a direction of linear velocity of the roller), these metal deposition ions are separated on the scalp by a distance that is comparable to the inter-electrode distance illustrated in FIG. 2.

As noted above, in some embodiments, alternating discs are zinc-electrodes and alternating discs are copper-electrodes. According to this non-limiting example, all electrodes 102 of discs 104A, 104C, and 104E deposit a cation (e.g. zinc) and all electrodes 102 of discs 104B, 104D and 104E deposit an anion.

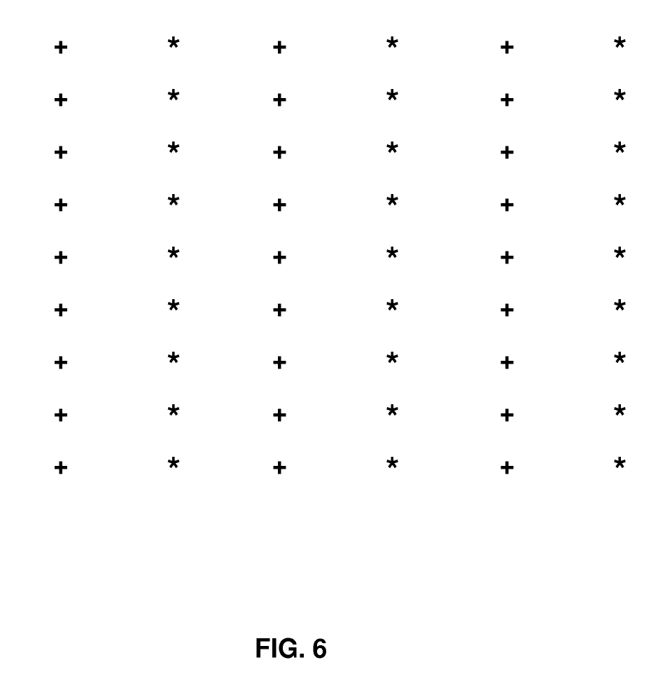

FIG. 6 schematically illustrates metal-ion-deposition islands on the scalp after rolling such a device over a user's scalp. In the schematic example of FIG. 6, cation metal-ion-deposition islands are represented as “+” (plus) while anion metal deposition islands are represented as “*” (star). In this example: (i) a distance between adjacent deposition islands of the same polarity (i.e. a distance between two neighboring pluses, or between two neighboring stars) is approximately equal to an inter-electrode distance for electrodes 102 disposed along a circumference of a disc; and (ii) a distance between deposition islands of opposite polarity (i.e. a distance between a neighboring star and plus) is approximately equal to a lateral distance between laterally-adjacent discs

FIG. 6 relates to the situation of a ‘single pass’—i.e. the roller is moved in a single linear direction over the scalp. In some embodiments, the roller may be moved back and forth′ to perform a ‘multi-pass’ treatment. For example, the roller may be manually moved, the user may not move the roller in exactly a straight line introducing some degree of randomness in the distances between neighboring deposition-islands.

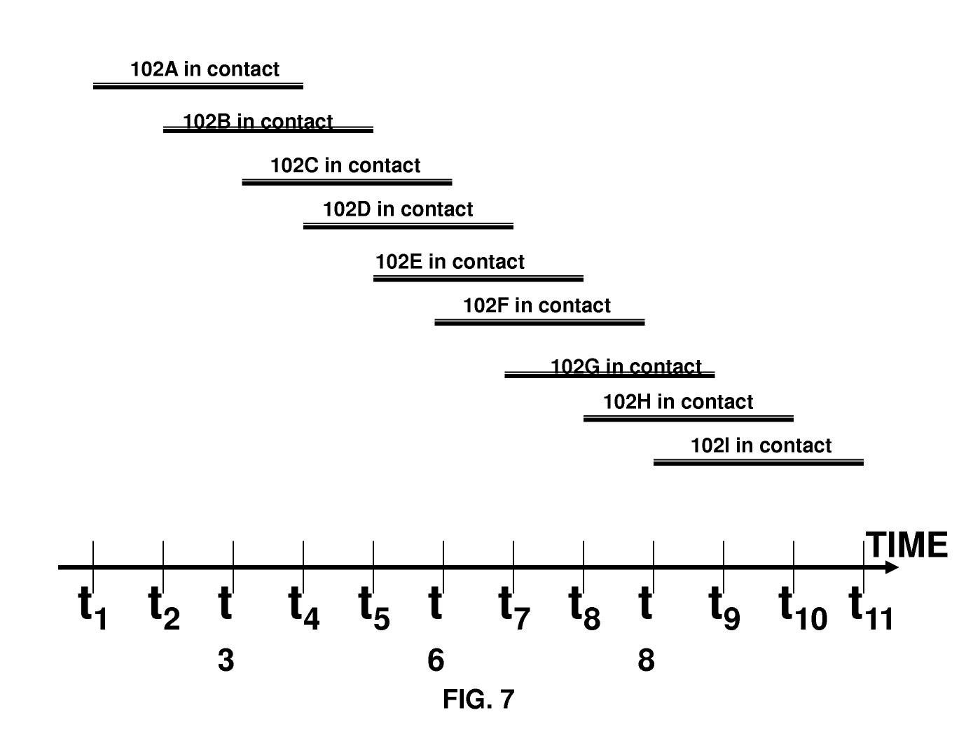

As illustrated in FIG. 5, in some embodiments, multiple electrodes of the same disc are simultaneously in contact with the scalp—in FIG. 5, electrodes 102H-102K are simultaneously in contact with the skin. FIG. 7 illustrates a timeline showing where electrodes 102A-102I are brought into contact and out of contact with the scalp—for example, electrode 102A is in contact with the scalp between times t1 and t4, electrode 102B is in contact with the scalp between times t2 and t5, and so-on.

When an electrode is in contact with the scalp, this is an ‘electrode-scalp contact events’—FIG. 7 illustrates the commencement and conclusion of electrode-scalp contact events for electrodes 102A-102I in a heuristic example. Typically and as discussed below, each electrode-scalp contact event is quite brief—for example, at most 100 milli-seconds. Nevertheless, the present inventors have found that even this very brief contact is sufficient to form a small metal-deposition island on the scalp, and that it is useful to form a large number of distinct metal-deposition islands, preferably, within a relatively short period of time.

It is possible to employ external electrical power to increase a current between electrodes of opposite polarity through the scalp while both electrodes are in contact with the skin, rather than relying exclusively on the galvanic current between electrodes. In some embodiments, this may allow for a therapeutically significant quantity of metal ions in the metal-deposition-island formed by each contact event selected from a plurality of contact events, despite the relatively short electrode-scalp contact period of each contact event.

In some embodiments, some but not all contact events cause deposition of metal ions on the skin or scalp. In these embodiments, it is still possible to discuss a feature of a specific set of contact events where all events are the specific set are metal-ion-depositing—however, it is understood that additional contact events may be performed before and/or after and/or after a time-frame of the ‘specific set of contact event.s’

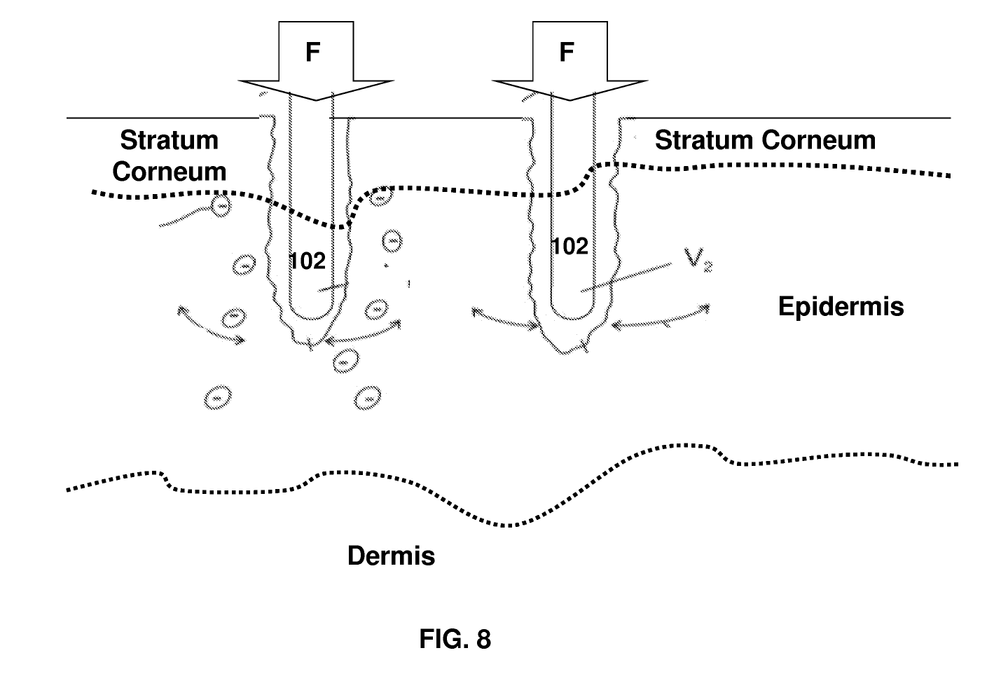

In the example of FIG. 8, the electrodes 102 are ‘non-wounding’ since they do not enter the dermis. The rounded tips of the electrodes allows the user to provide significant pressure (e.g. at 0.5 mega-Pascal) to achieve a less invasive but sufficiently-stimulating ‘micromassage’ effect rather than a wounding or dermis-penetrating effect.

In the example of FIG. 8, negatively-charged ions are deposited on the skin to create the metal-ion-deposition island.

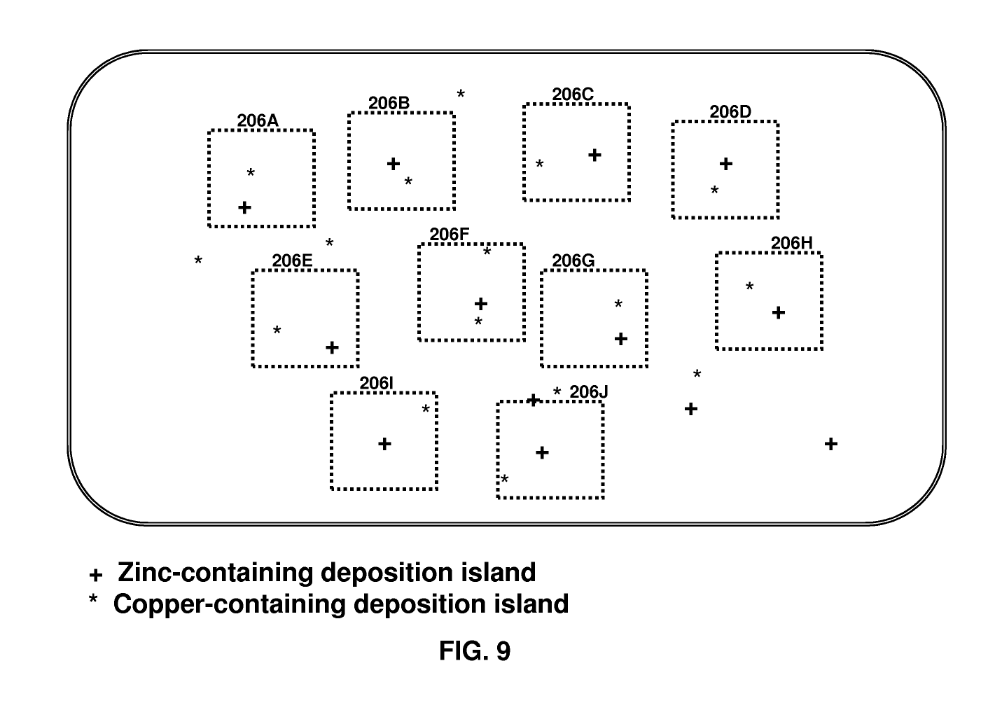

FIG. 6 illustrates on pattern of metal-ion-deposition islands. FIG. 9 illustrates another pattern. In the example of FIG. 9, a region of scalp comprises a plurality of different square ‘patches’ 206 (patches 206A-206J are illustrated) where a patch is a geometric construct to describe a portion of scalp. For example, a size of each scalp patch may be n mm X n mm where a n is a positive number having a value of at most 5. In the example of FIG. 9, cation and anion metal-deposition islands are both respectively applied to each patch of the ten patches.

Thus it may be said that at least one first-metal-deposition-island (i.e. represented by a ‘+’) and at least one second-metal-deposition-island (i.e. represented by a ‘*”) are both respectively and distinctly formed on each n mm x n mm scalp scalp-patch 206 selected from a 10-member scalp-patch sub-set of the scalp-patch set.—for example, the 10 member scalp patch set {206A,206B,206C,206D,206E,206F,206G,206H,206I,206J}.

The term ‘metal-ion-deposition’ island refers to deposition of metal on the user's scalp such that at the moment of deposition, the metal is deposited as an ion. There is no requirement for the metal to remain in ionic form thereafter. A metal-ion-deposition island forms a localized portion of metal on the user's scalp.

Examples described above relate to deposition by a multi-disc roller. Alternatively or instead of using disks, the electrodes may protrude from a single solid roller (e.g. spherical or cylindrical). In one example, electrodes are disposed at different longitudinal positions along the roller. As discussed below, the method may be performed using a non-roller device.

Also illustrated in FIG. 1 are axle 112, handle 114, housing 110, and power-source 116.

Although some electrode-scalp contact events form metal-deposition-islands, not every contact event is required to deposit metal on the user's scalp.

Example Performance Parameters

One non-limiting use case relates to the following parameters: (i) a disc radius of 16 mm and circumference of about 100 mm; (ii) about 100 protrusions per disc so that a distance between neighboring protrusions along a disk circumference is about 1 mm; (iii) the user applies pressure (e.g. at least 0.5 mega-Pascal or at least 1 mega-Pascal per electrode) has he/she rolls the disc array over his/her scalp, and thus rolls the disc area at a rate of about 0.3 revolutions/second corresponding to a linear velocity, assuming

Assume a 2-disk device, the number of distinct contact events per second (i.e. where a protrusion is brought into and out-of contact with the scalp) in this example is about 0.3*100*2≈65 contact-events per second. In this situation, assuming the user continuously rolls the disc over his/her scalp for at least one minute, the scalp would be subjected to about 4000 electrode-scalp contact events per minute.

Assuming an 8-disk device, the user's scalp would be subjected to about 16,000 contact events per minute.
Cross sectional area of individual electrode-scalp contact-location and/or metal-deposition island: In an exemplary embodiment of the invention, the cross sectional area of an electrode-scalp contact location is selected to be, for example, about 1 mm², about 0.1 mm², about 0.01 mm², about 0.001 mm², about 0.0001 mm², or other smaller, intermediate or larger sizes are used.
Density: In an exemplary embodiment of the invention, the density of contact locations and/or deposition islands per unit area of scalp to be treated is selected, for example, about 1 locations/mm², about 5 locatinos/mm², about 8, locations/mm² about 10 locatinos/mm², or other smaller, intermediate or larger densities are used.
Total electrode-scalp contact area per electrode per contact event: In an exemplary embodiment of the invention, the area of scalp to be subjected to ion-deposition from the total area of the scalp to be treated is selected. The ‘fill factor’ is selected to be, for example, about 10%, about 1%, about 0.1%, about 0.01% of the area to be treated, or other smaller, intermediate or larger values are used. In one non-limiting example, the fill factor is (i) at least 5% or at least about 7.5% and/or (ii) at most 50% or at most 40% or at most 30% or at most 20% or at most 15%.
Gaps between deposition islands: In an exemplary embodiment of the invention, the distance between deposition-islands is selected. Optionally, the space between deposition-islands along a first axis is selected. Optionally or additionally, the space between deposition-islands along a second axis is selected, for example, the first and second axes are perpendicular to one another. In some embodiments, gaps along at least one axis are selected according to the existing amount of hair at the area to be treated, for example, relatively larger spaces are selected for a region with relative denser hair and/or hair having a relatively larger diameter. Existing hair may be displaced to the gaps between the deposition-islands. Spaces between deposition islands along the first axis are selected to be about, for example, 3 mm, about 4.5 mm, about 6 mm, or other smaller, intermediate or larger spaces are used. Spaces between electode deposition islands along the second axis are selected to be, for example, about 0.3 mm, about 0.5 mm, about 1 mm, about 1.5 mm, about 2 mm, or other smaller, intermediate or larger values are used

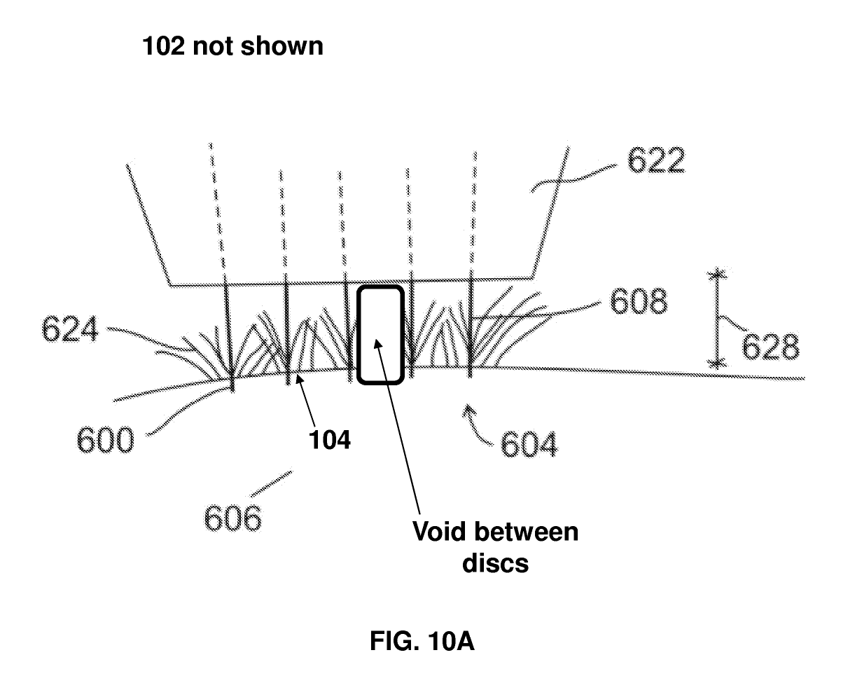

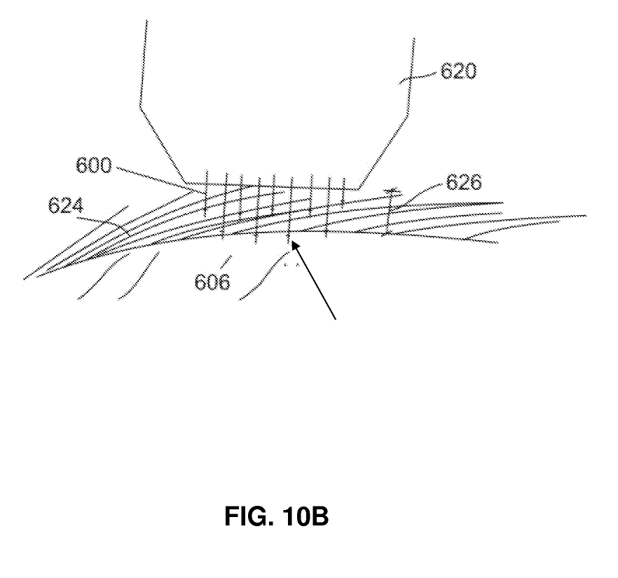

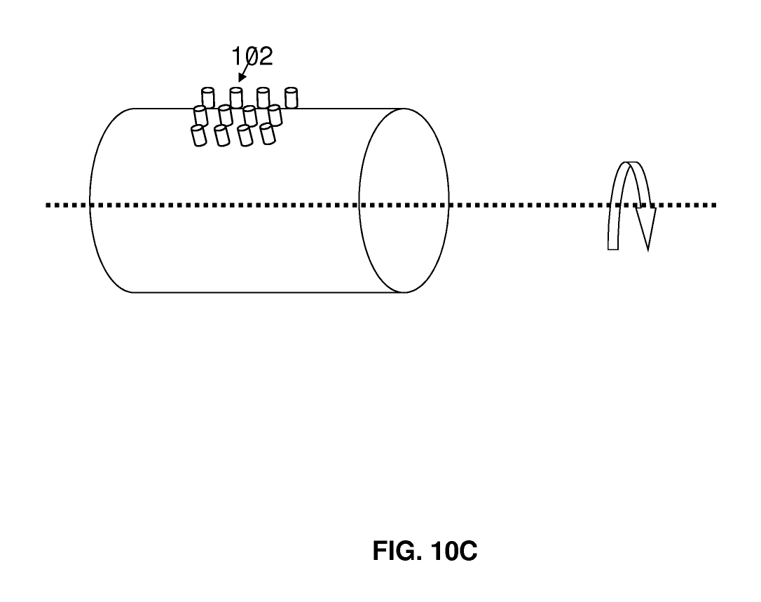

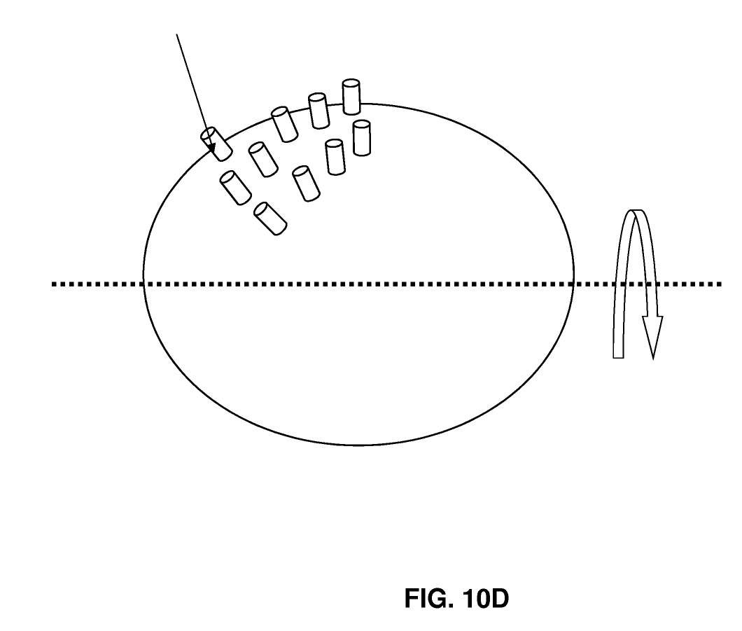

FIG. 10A-10B are sides view of a electrode array 604 using electrodes 600 to cause a pattern of deposition islands in the scalp 606.

Also illustrated in FIG. 10A are voids between disc—this allows the discs to penetrate through the hair 624—i.e. when the discs penetrate through the hair, the hair is located in the voids between the discs.

In an exemplary embodiment, an actuator moves the electrode up and/or down.

In some embodiments, a group of electrodes is attached to a single actuator.

In an exemplary embodiment of the invention, a distance 626 and/or 628 between scalp 606 and device head 620 and/or 622 is set to provide a volume for hair 624 during penetration of electrodes 600 through the hair to contact scalp 606. Hair 624 can be displaced into the volume to let electrodes 600 contact scalp 606 to allow the full length of electrodes 600 to enter. Distance 628 can be set for example, by diameter of discs 608 and/or by selecting the central hinge position within device head 620.

In an exemplary embodiment of the invention, the pattern of deposition-islands is parallel straight lines, for example, for a roll of discs 608. Optionally, complex and/or random patterns of deposition islands can be created by repeated rolling of discs 608 over the scalp. Optionally, one or more discs each comprise multiple electrodes, arranged, for example, in a circumferential arrangement and/or along the thickness of the wheel, on the surface contacting the skin.

In an exemplary embodiment of the invention, electrodes 600 are made out of a biocompatible material, non-limiting examples include; metals (e.g., steel, silver, gold), alloys, glass, plastic, ceramic.

In an exemplary embodiment of the invention, electrodes 600 are coated with a type I 5a-reductase inhibitor, for example the metals zinc and/or copper.

It is noted that a series of discs disposed along a common rotation axis (see FIG. 2, 10A) is just one example of a ‘roller’ having protrusions extending therefrom (e.g. ion-releasing and/or electrode-protrusions). For the case of the series of discs, each disc has substantially the same diameter so that the protrusions extended radially/outwardly from a common ‘geometric-construct cylinder’ that, for example, rotates at a common rotation rate (e.g. individual discs rotate in-tandem). This the series of discs is one example of a ‘cylindrical roller.’

Another example of a cylindrical roller is illustrated in FIG. 10B—typically the protrusions would be distributed around a circumference of the roller as was the case for the disks—the fact that only a few protrusions as illustrated in FIG. 10C is for brevity, and is not meant to represent the typical case.

A cylindrical roller is one example of a ‘round roller’—other examples may be a spherical roller shaped like an ‘American football’ illustrated in FIG. 10D.

### Electrode/Protrusion Actuators

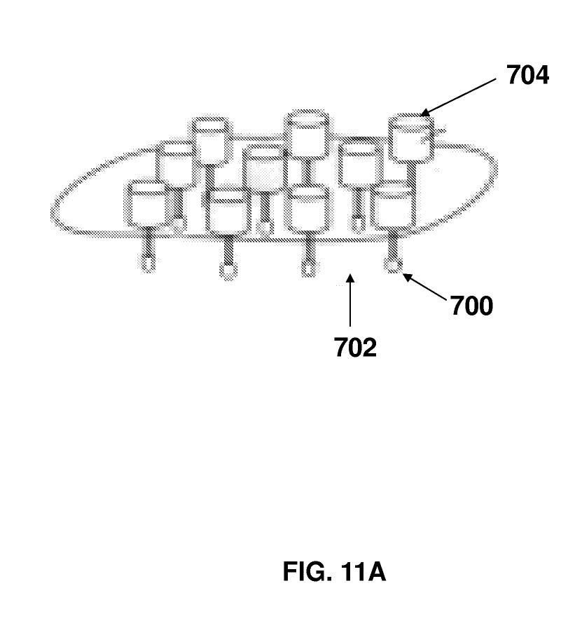

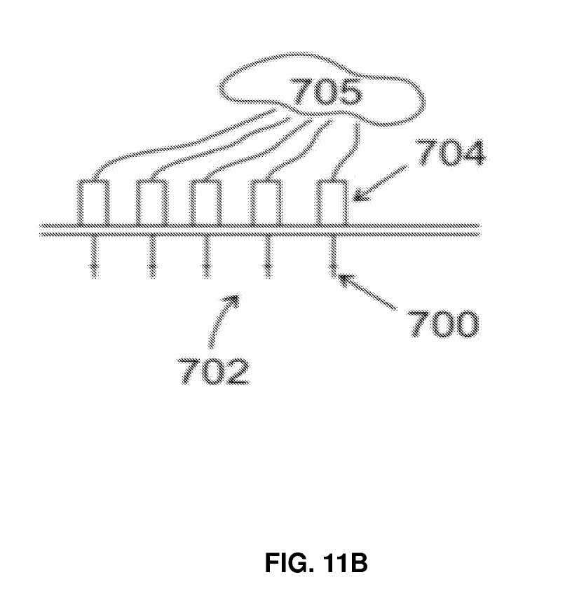

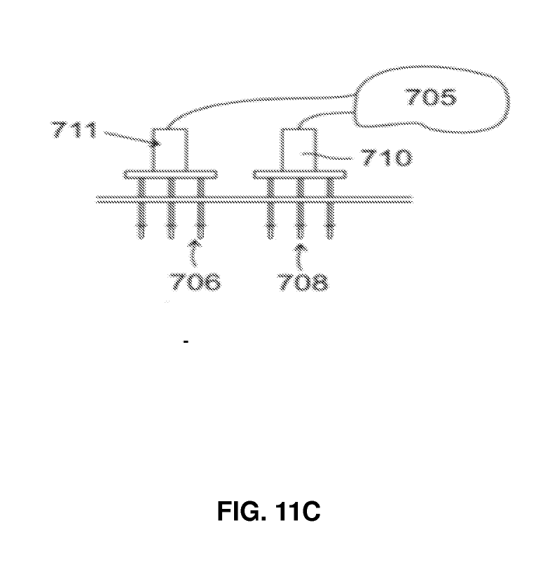

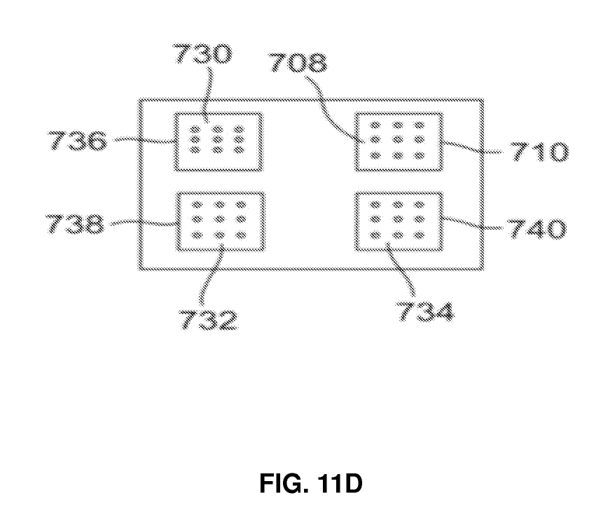

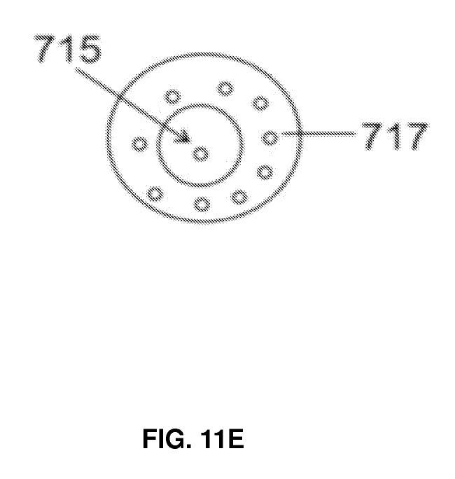

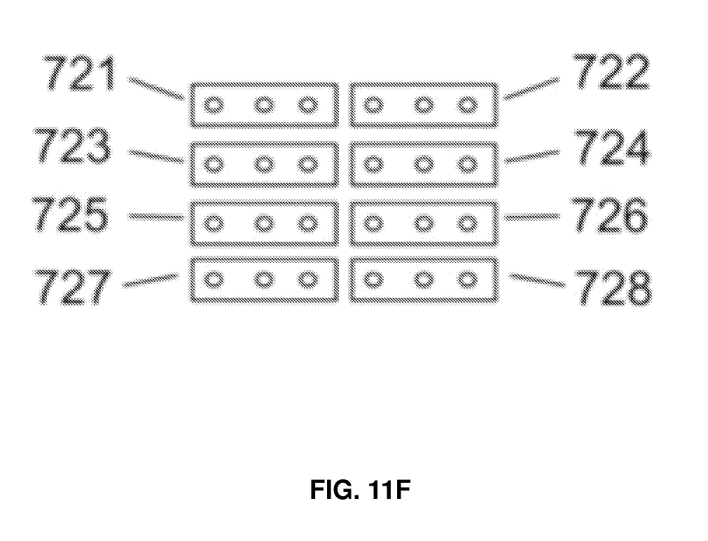

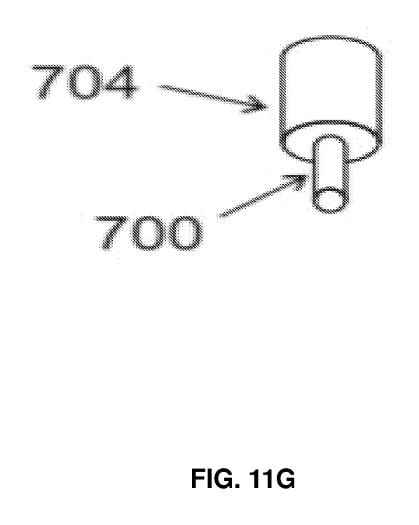

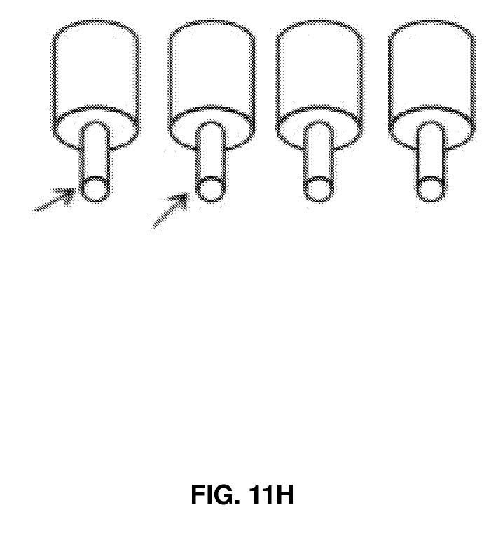

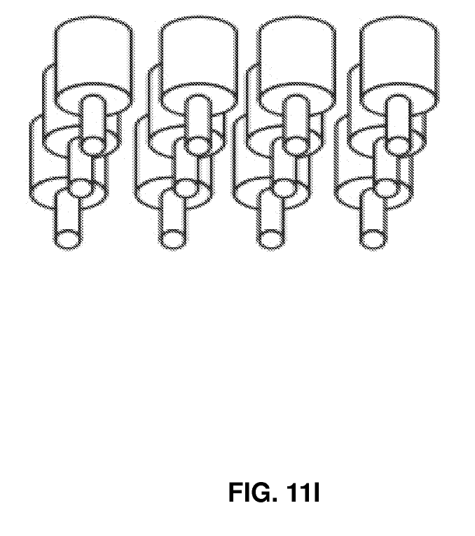

FIGS. 11A-11F are illustrations of embodiments of electrode actuators, in accordance with some embodiments of the invention. Optionally, electrode actuators act as vibrational elements, to vibrate electrodes according to the selected vibrational protocol.

In some embodiments of the inventions, one or more non-limiting examples of actuators include; piezoelectric elements, motorized linear actuators, and/or shape memory alloy actuators.

In some embodiments of the invention, electrodes are individually vibrated. Alternatively or additionally, groups of electrodes are vibrated together. Optionally, vibration is performed by an off-axis spinning mass, for example, the direction of the axis determines the plane of vibration. For example, translating the movement to a linear direction, pushing on a piston mass creates a linear vibration.

FIG. 11A is an isometric view, and FIG. 11B is a cross sectional view of a electrode array 702, for example described with reference to FIG. 10B. Each electrode 700 (of array 702 is coupled to an actuator 704. Optionally, each electrode 700 is coupled to a separate actuator 704. Optionally, actuators 704 are attached to a power control 705.

For example, the actuators 704 may be controlled to maintain the electrode in contact with the scalp for only brief electrode-scalp contact events.

FIG. 11C is an isometric view, and FIG. 11D is a cross sectional view of a electrode array 706. Two or more electrodes are controlled by actuators, for example, array of nine electrodes 708 is controlled by actuator 710 and, for example, array of electrodes 706 is controlled by actuator 711. There are two or more groups of electrodes, for example, four groups 708, 730, 732 and 734 of nine electrodes in each group are controlled by four actuators 710, 736, 738 and 740.

Electrode groups can be arranged in a variety of patterns. Non-limiting examples include the checkerboard pattern as illustrated in FIG. 11D, a bull's eye pattern as illustrated in FIG. 11E and/or a side by side tile pattern as illustrated in FIG. 11F. For example, the bull's eye pattern (FIG. 11E) may comprise one electrode 715 in an inner circle and at least two electrodes in electrode array 717 in an outer circle and, for example, the side by side tile pattern (FIG. 11F) may comprise eight groups 721, 722, 723, 724, 725, 726, 727 and 728 of electrodes.

In some embodiments, at least two groups (FIG. 11F) may touch the scalp simultaneously. For example, the device is configured so that several actuators receive a signal to “lower” and touch and/or penetrate the scalp simultaneously. Optionally or alternatively, several electrodes are connected to a single actuator 710 and go up and down together. Optionally, the electrodes conform (or are advanced to conform) to the scalp curvature and penetrate together. In some embodiments, the electrodes are equipped with a spring to facilitate conformity to the scalp curvature.

In an exemplary embodiment, 721 and 722 may touch the scalp simultaneously, 722 and 723 may touch the scalp simultaneously, or 723 and 724 may touch the scalp simultaneously, or 724 and 725 may touch the scalp simultaneously, or 725 and 726 may touch the scalp simultaneously, or 721, 722 and 728 may touch the scalp simultaneously, or 722, 725 and 727 may touch the scalp simultaneously or another combination of groups may touch the scalp simultaneously. Optionally, more than two types of ions are discharged from the electrodes. FIG. 7G is an isometric view of a single injector.

FIG. 11H is an isometric view of a 1-dimensional array of electrodes. FIG. 11I is an isometric view of a 2-dimensional array of electrodes.

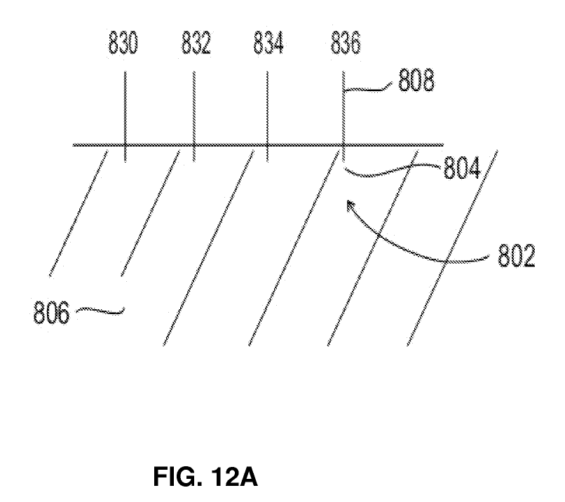

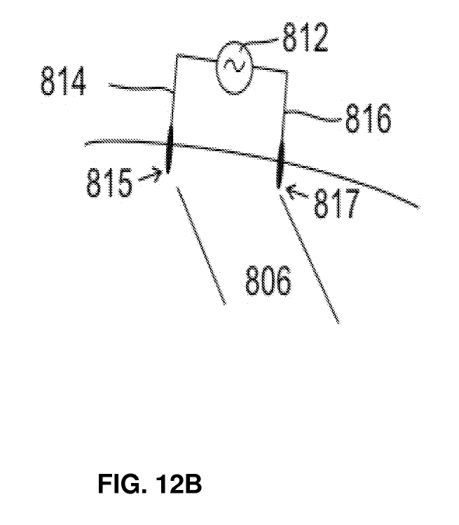

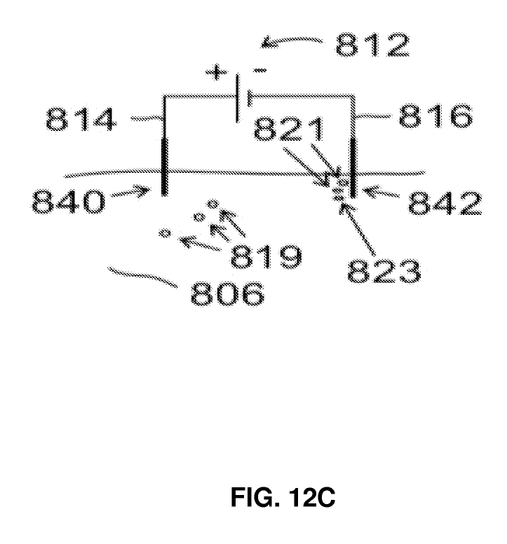

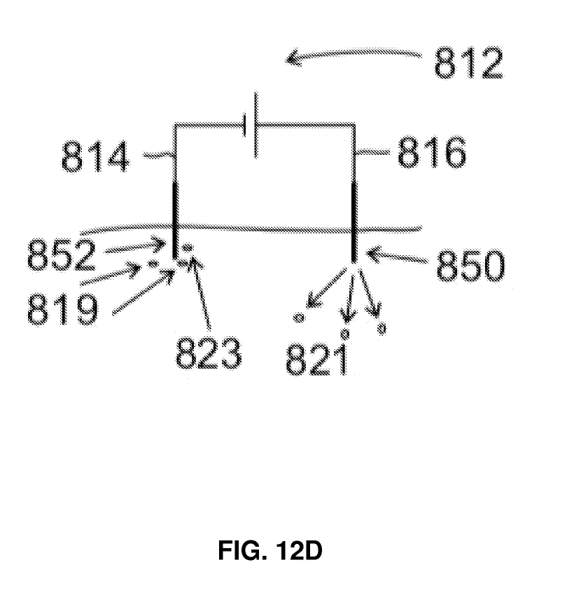

Reference is now made to FIGS. 12A-12D. In some embodiments, the number of ions deposited during treatment is controlled by adapting the voltage (see, for example, the methods described in Chizmadzhev et al, Electrical Properties of Skin at Moderate Voltages: Contribution of Appendageal Macropores), by adapting the temperature (see, for example, the methods described in Maulsby et al, The interrelationship between the galvanic skin response, basal resistance, and temperature), and/or by adapting the frequency. Increasing the voltage, temperature and frequency can each increase the number of ions deposited. For example, the number of ions deposited during treatment is controlled in an open loop manner by determining the voltage before beginning treatment. Alternatively, the number of ions deposited during treatment is controlled in a closed loop manner by determining the voltage during the treatment based on feedback received from sensors incorporated into the device.

In some embodiments, controlling the ions deposited is done directly by measuring the charge of each polarity (ion type) or of both, for example, by measuring and integrating the (absolute) current passed through each type of disk set or through both. The existence of current indicates the unit is in actual use. A degradation of current indicates a faulty unit, improper contact, or other means. Excessive current might indicate a faulty unit, or excessive moisture on the scalp (and therefore not enough current through the scalp).

In some embodiments, the mass of metal ions discharged from the electrodes may be calculated by a formula. For example, assuming the charge C is ionic, and the oxidation state Z, the mass m of metal ions discharged from the electrodes (w is the atomic mass, e the electron's charge, Na is Avogadro's number) is computed as follows:

$$m = \frac{C - w}{e - Z - {N}_{a}}$$

In some embodiments, ion injecting electrodes that touch the scalp are connected to one terminal of a power source and an electrode that does not touch the scalp is connected to a second terminal of the power source. For example, the electrode that does not touch the scalp may be connected to a part of the body other than the scalp. For example, the device may comprise a handle comprising an electrode designed to touch the palm of a person holding the handle.

In some embodiments, the efficiency of the deposition of ions is enhanced, for all users or for a specific user, by performing a “calibration phase” in which the same region is treated for a period of a time while changing each parameter slightly and measuring the real-time response in current. Optionally, different treatment parameters may be chosen for different scalp areas of same user. Optionally, different treatment parameters may be chosen for different users.

In some embodiments, the efficiency of the deposition of ions is enhanced through general improvements in the parameters, for example, preparing a better cross section of the electrodes and/or starting with more efficient voltage and frequency. Optionally, the efficiency of the deposition of ions is enhanced through dynamic modification of changeable treatment parameters through closed-loop feedback/control.

In some embodiments, ion penetration increases blood flow when the electrical fields generated by the small charge deposits create a MENS (microcurrent electrical neuromuscular stimulation) effect in the skin. Optionally, the MENS effect shortens skin healing times. Optionally, the electrical fields invigorate movement of essential ions and stimulate the skin systems into an increased rate of activity.

FIG. 12A is an illustration of an array of electrodes 802 depositing materials 804 beneath the skin 806 surface of scalp, in accordance with an exemplary embodiment of the invention. For simplicity purposes, array 802 comprises four electrodes 808, having the material 804 to deposit located at the part of the electrode 808 that contacts scalp 806.

In an exemplary embodiment of the invention, electrodes 808 are made of material 804. Alternatively, electrodes 808 are coated with material 804. Optionally or alternatively, 830, 832, 834 and/or 836 represent electrical potentials which may exist on electrodes 808.

In an exemplary embodiment of the invention, two different electrodes 808 to be electrically coupled have two different materials 804 at their ends. For example, alternating discs (e.g., as illustrated in FIG. 1) are made from different materials, for example, copper and zinc.

In some embodiments, scalp 806 acts as a bridge, placing two electrodes having dissimilar metals in electrical contact. The metals can undergo galvanic corrosion, where one metal dissolves in scalp 806, while the other metal absorbs ions from scalp 806. For example, if one metal is zinc and the other metal is copper, the zinc will dissolve and the copper will accumulate. Optionally, material 804 is chosen to have other depositing effects. Optionally or additionally, current is forced in the opposite direction.

FIG. 12B is an illustration of ion deposition into scalp 806 for example using a galvanic cell set-up, in accordance with an exemplary embodiment of the invention. Optionally, a power source 812 electrically couples a first electrode 814 and a electrode electrode 816. For example, each electrode 814 and 816 may be coated electrodes comprising different materials at the ends 815 and 817, for example, electrode 814 touches scalp 806 at end 815 with zinc and electrode 816 at end 817 with copper. Optionally, power source 812 emits Alternating Current (AC). Optionally, power source 812 emits Direct Current (DC).

FIG. 12C is an illustration of using the set-up as in FIG. 12B to release zinc ions into scalp 806, in accordance with an exemplary embodiment of the invention. The positive pole of power source 812 is electrically connected to electrode 814 with zinc (e.g., acting as the anode 840), and the negative pole is electrically connected to electrode 816 with copper (e.g., acting as the cathode 842) Zinc ions 819 are discharged from electrode 814 into scalp 806, and copper ions 821 and/or other ions 823 are accumulated from scalp 806 onto electrode 816.

In an exemplary embodiment of the invention, the voltage of power source 812 as in FIG. 12C is, for example, about 1V, about 3V, about 5V, about 7V, about 10V, about 30V, or other smaller, intermediate or larger values are used.

FIG. 12D is an illustration of using the set-up of FIG. 12B to release copper ions into scalp 806, in accordance with an exemplary embodiment of the invention. The positive pole of power source 812 is electrically connected to electrode 816 with copper (e.g., acting as the anode 850), and the negative pole is electrically connected to electrode 814 with zinc (e.g., acting as the cathode 852). Copper ions 821 are discharged from electrode 816 into scalp 806, and zinc ions 819 and/or other ions 823 are accumulated from scalp 806 onto electrode 814.

In an exemplary embodiment of the invention, the voltage of power source 812 as in FIG. 12D is at least greater than the standard potential for the reaction, for example, above 1.10 Volt.

In an exemplary embodiment of the invention, power source 812 is an alternating current source. The frequency of source 812 can be selected to result in a desired ion deposition pattern, for example alternating between the set-ups as described in FIGS. 12C and 12D. For example, the frequency of source 812 is selected to be substantially half of the rate of electrode-scalp contact events per second, for example when using the hair stimulation device with rolling discs, for example, as described with reference to FIG. 1. For example, if the device is rolled over the scalp to achieve a rate of scalp-electrode contact events of 30 events per second and the frequency of source 812 is 15 Hz, the ions deposited during each electrode-contact will alternate, for example between copper and zinc. Furthermore, different ions will be deposited at different locations.

In some embodiments of the invention, the AC waveform (e.g., duty cycle) is selected according to the ratio of the desired material deposition. For example, to achieve a 10:1 ratio (e.g., of zinc:copper), a waveform having a 10:1 ratio (91% duty cycle) is selected. Alternatively or additionally, the number of electrodes coated with each material is selected according to the desired deposition ratio, for example, the number of electrodes coated with zinc relative to the number of electrodes coated with copper is 10:1.

In some embodiments of the invention, power source 812 is a direct current source. The polarity of source 812 can be selected to result in a desired ion type and/or deposition pattern. For example, according to the set-ups of FIGS. 12C and/or 12D. The set-up of FIG. 12C can also be achieved without source 812, for example by electrically connecting electrodes 814 and 816.

In some embodiments of the invention, materials (e.g., ions) are added directly to the scalp, for example in the form of a lotion, gel and/or water. Non-limiting examples of ions in this form include ZnSO₄, CuSO₄. The lotion can be added in addition to the use of coated electrodes, or instead of coated electrodes (e.g., using uncoated electrodes). Optionally, the ions penetrate below the surface of the skin.

### Electrical Stimulation

In an exemplary embodiment of the invention, the scalp is stimulated by applying one or more currents and/or voltages to areas of the skin, for example, an electrical stimulation protocol is selected. Optionally, a plurality of currents and/or voltages are applied to the scalp, for example different voltages and/or currents to different areas and/or between different electrodes.

In an exemplary embodiment of the invention, the electrical stimulation is separate from the current applied to the electrodes to release ions, for example, Optionally, electrical stimulation is applied by one or more discs and/or electrodes, and ion deposition is applied by different discs and/or electrodes. Optionally, the electrodes to apply electrical stimulation but not ion deposition are inert, for example, made from platinum. Alternatively, a voltage is applied to the electrodes to prevent ion deposition by the galvanic effect. Alternatively or additionally, electrical stimulation and ion deposition overlap, for example, applied by the same discs and/or electrodes.

Inventors hypothesize that selectively applying a plurality of electrical stimulation patterns (e.g., voltages and/or currents) to the scalp will promote hair growth. However, the efficacy of some embodiments of the invention can be unrelated to the underlying theory, and work even if the theory is incorrect.

In an exemplary embodiment of the invention, the electrical stimulation protocol comprises one or more variables. Non-limiting examples of selectable parameters include:
Geometric voltage and/or current distribution pattern: The pattern of applied voltages and/or current per electrode. For example, the voltage and/or current at each electrode is independently controlled and/or groups of electrodes have similar voltages and/or current (e.g., alternating electrodes have similar voltages and/or currents, electrodes having the same type of material (for example zinc or copper) have similar voltages and/or currents).
In some embodiments of the invention, the voltage and/or current pattern is substantially the same, for example, the same electrode is associated with the same charge and/or current. Alternatively or additionally, the voltage and/or current pattern is dynamic, for example dynamic throughout the array, and/or a region of the array. For example, in a relatively large array, a relatively small patch of the electrical pattern can be scanned across the array.
A potential advantage of two groups of electrodes with different voltages is the controlled patterning of current and/or ion deposition. For example, local stimulation may be superior to global. Potentially, division to several groups allows greater flexibility and/or controllability of the current. For example, current can be applied (e.g., to different groups, at different intensities) simultaneously or in a time-divided manner.
Voltage and/or current distribution pattern over time: The pattern of applied voltage and/or current per electrode can vary over time. For example, an alternating current and/or voltage can be applied to vary the voltage and/or current between two or more electrodes (or groups of electrodes). In the case of using the device with discs for example in FIG. 1 (e.g., rolling the discs with electrodes on the scalp), selecting an alternating frequency that is less than the frequency of rotation can result in increasing the diversity and/or gradients of voltages and/or currents applied underneath the skin surface. Inventors hypothesize that applying various patterns of voltage and currents to the skin stimulates hair growth. Potentially, applying varying time and/or location stimulations improves stimulation of local points, for example hair follicles
Direct current (DC) offset: A voltage offset can be applied to the pattern applied to one or more electrodes. In an exemplary embodiment of the invention, the DC offset is calibrated, for example, from −3 volts to +3 volts, or other smaller, intermediate or larger values are used. In an exemplary embodiment of the invention, the DC disc to disc relative voltage ranges, for example, from 0 to 30 volt, or other smaller, intermediate or larger values are used.
Alternating current (AC) peak to peak voltage: In an exemplary embodiment of the invention, the peak to peak voltage of the AC varies, for example, from −10 volts to +10 volts, or other smaller, intermediate or larger values are used.
Frequency of AC: In an exemplary embodiment of the invention, the frequency of AC ranges, for example, from 10-1000 Hz, or other smaller intermediate or larger values are used.
Waveform of AC: In an exemplary of the invention, the waveform of AC is rectangular. Alternatively, other waveforms are used, non-limiting examples include sinusoidal, triangular, sawtooth.
Maximal Current: In an exemplary embodiment of the invention, the total electrical current is less, for example, than 0.5, less than 1, less than 2 milliAmperes, or other smaller, intermediate or larger values are used.

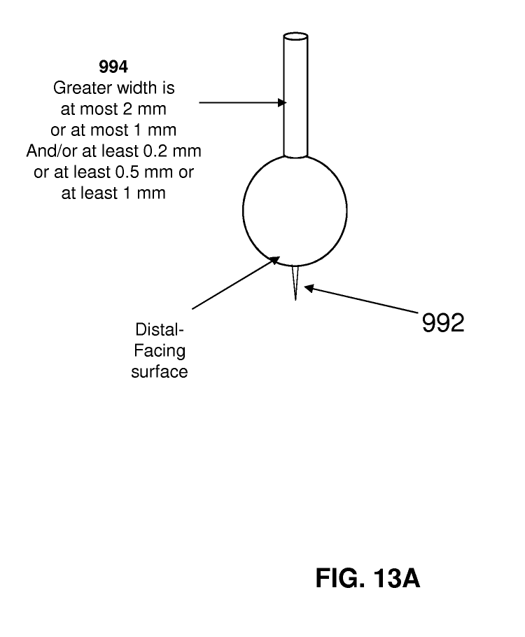

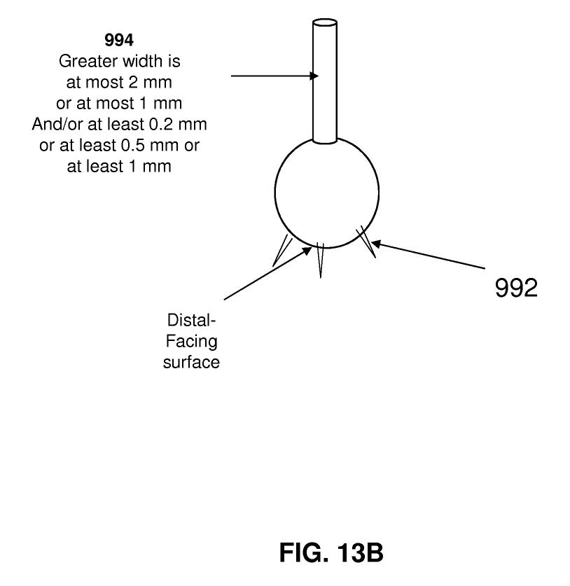

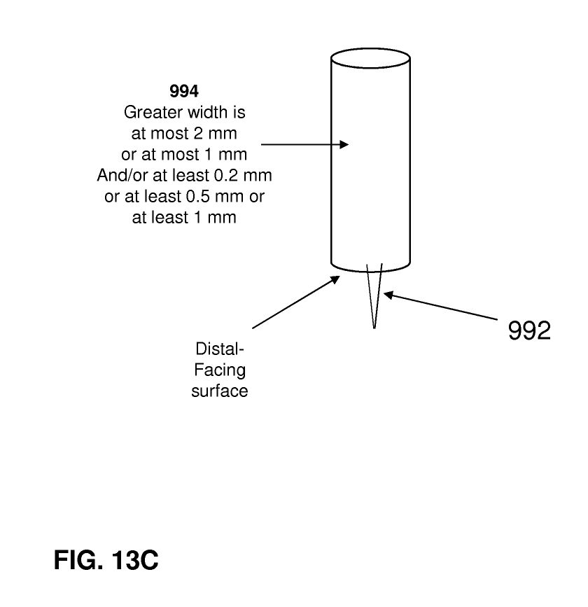

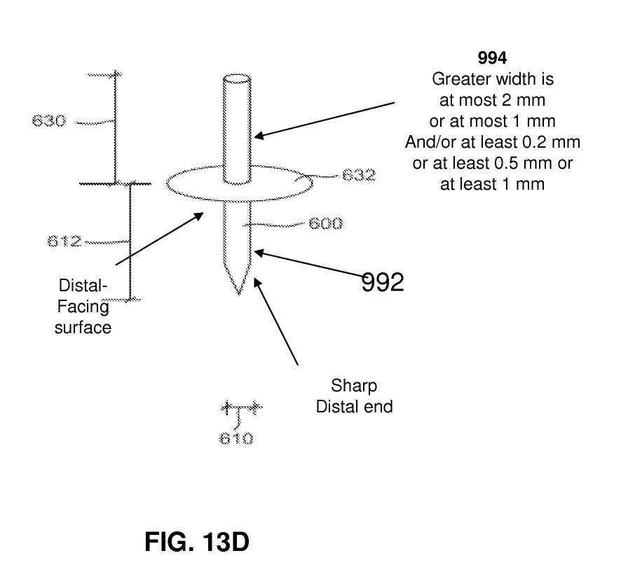

### A Discussion of FIG. 13A-13D—Protrusions Designs to Penetrate Only a Short Depth of the Skin

In some embodiments, the protrusions 102 are designed to regulate a depth of skin-penetration during use—e.g. so the skin is penetrated to a depth of least 5 microns or at least 10 microns and/or at most 100 microns or at most 75 microns or at most 50 microns or at most 20 microns. This may be the penetration during ‘ordinary use’ and/or when a tip of the electrode-protrusion is pressed against a healthy human scalp at a pressure of between 0.1 to 5 MPa (e.g. a pressure of about 0.1 MPa or a pressure of about 0.5 MPa or a pressure of about 2 MPa or a pressure of about 3 MPa or a pressure of about 4 MPa or a pressure of about 4 MPa or a pressure of about 5 MPa)

For example, this may be at a localized electrode-scalp contact area of at mots 10 mm̂ 2

Examples of protrusions having this capability are illustrated in FIG. 13A-13D.

A ‘greater thickness’ refers to cross-section of the protrusion—there is a thickness in two orthogonal directions (i.e. orthogonal to each other and perpendicular to the longitudinal direction)—the ‘greater thickness’ is the greater of these dimensions.

In some embodiments, each of the protrusions compriss: (i) an electrode-protrusion main body 994, characterized by a greater-thickness of at most 2 mm (e.g. at least 0.5 mm or at least 1 mm) and/or a length at least 0.2 mm or at least 0.5 mm or at least 1 mm, the main-body being blunt at its distal end and/or the main-body having a blunt distal-facing surface; and one or more sharp mini-needle(s) 992 extending from the blunt distal end or the blunt distal-facing surface of the main body, the mini-needle 992 being sharp at a distal surface thereof. In some embodiments, a length the sharp mini-needles is at least 10 microns or at least 20 microns and/or at most thereof being between 10 and 150 microns.

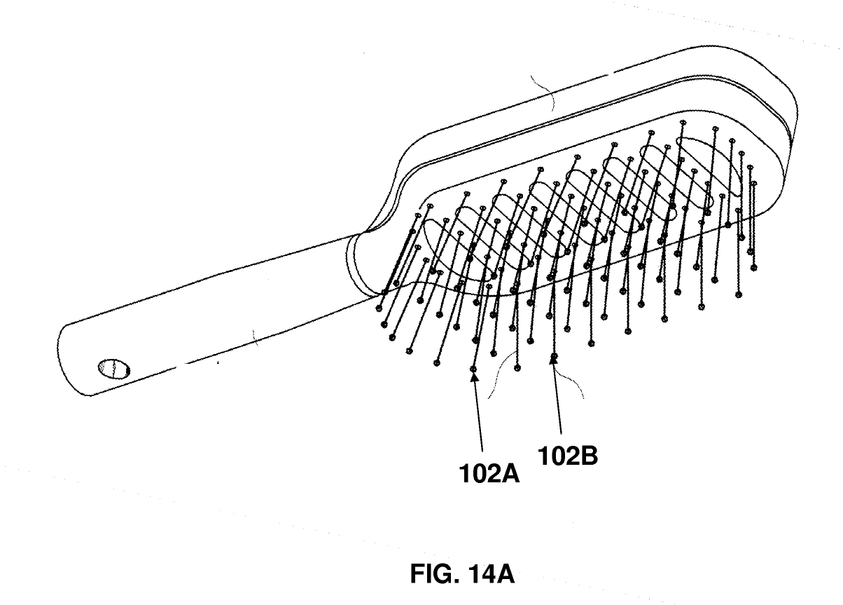

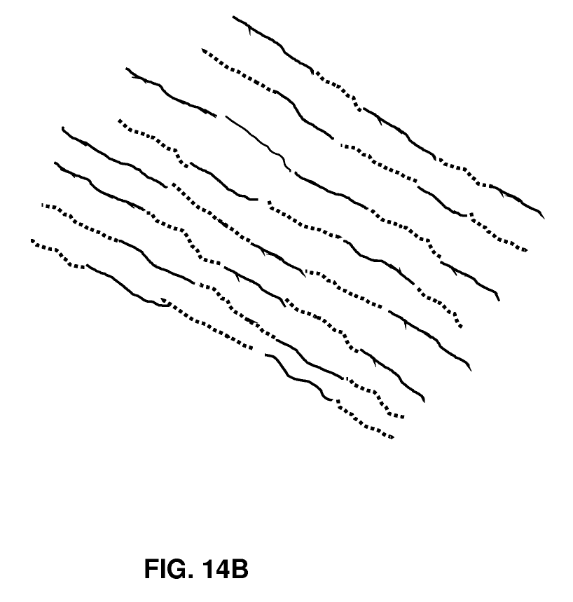

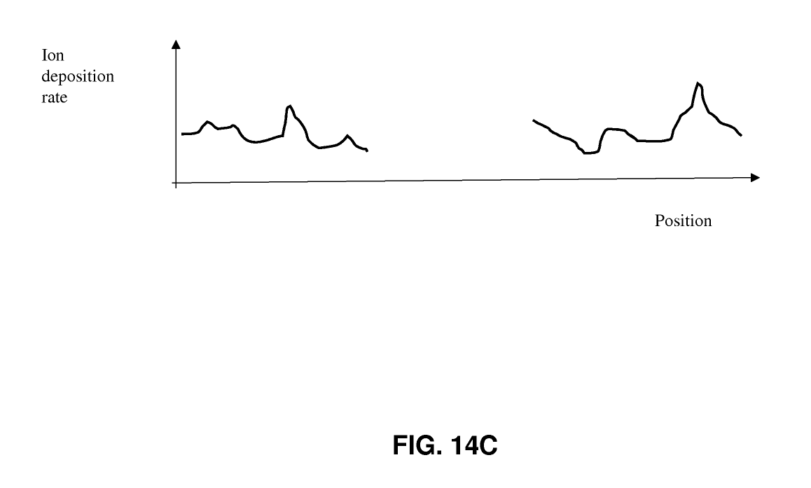

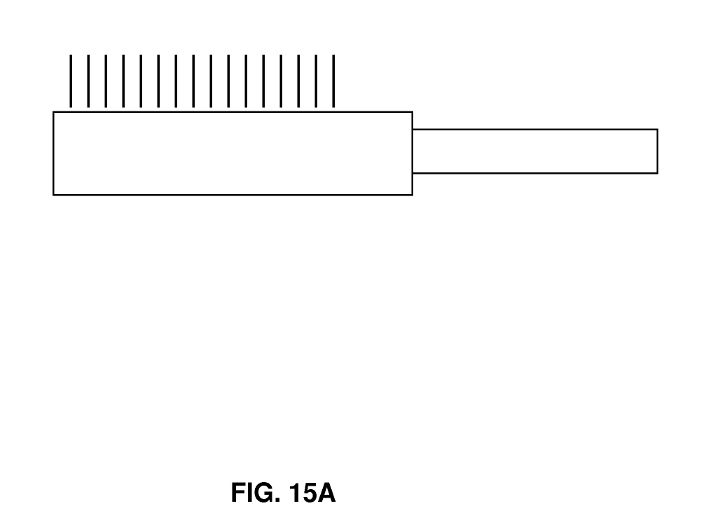

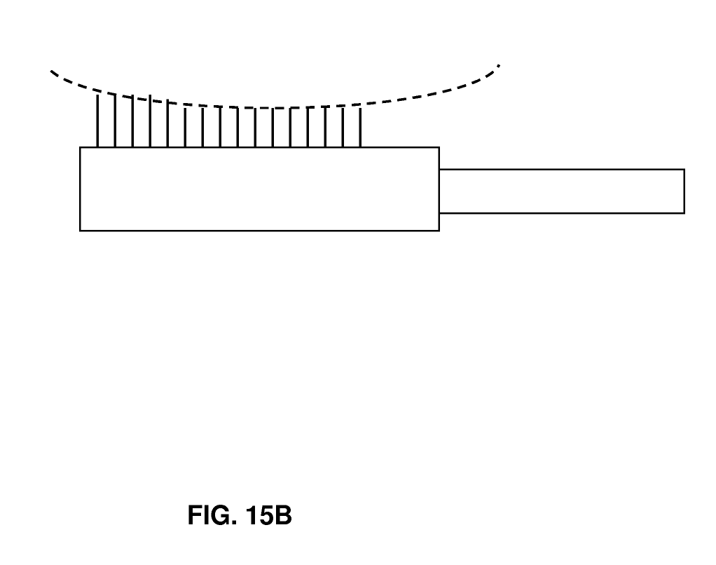

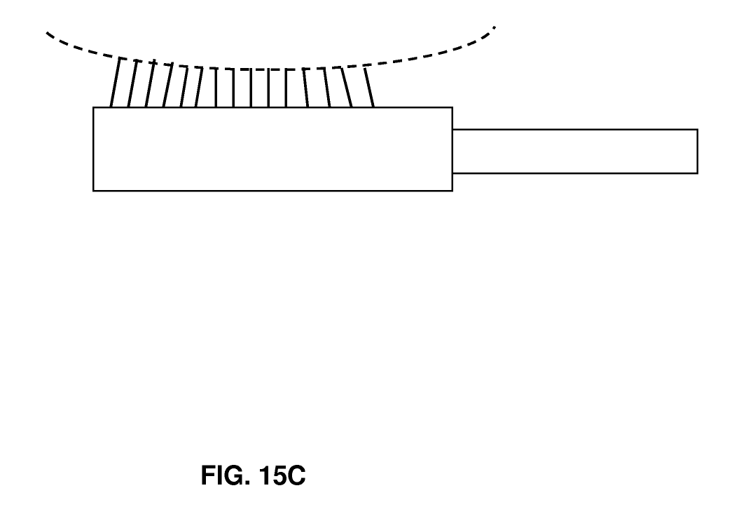

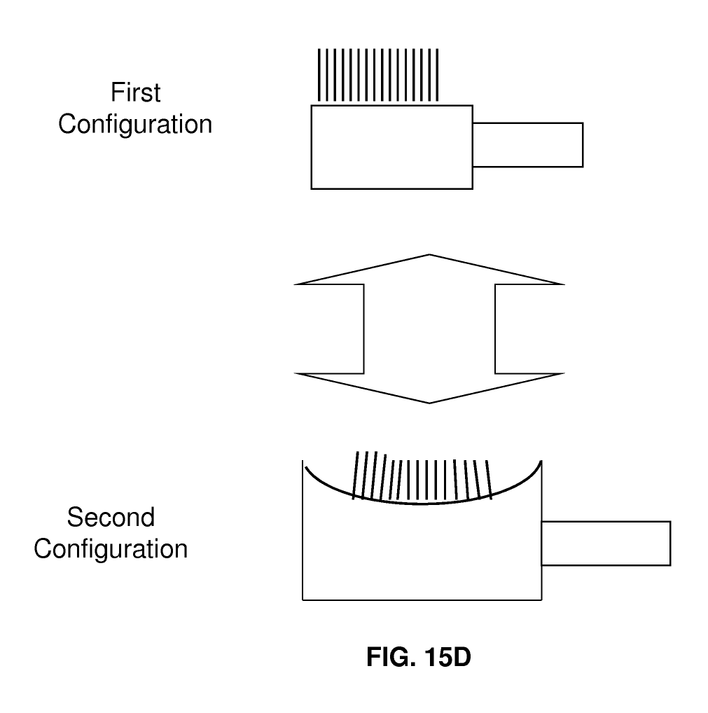

### A Discussion of FIGS. 14-15

In some embodiments, instead of a roller a scalp-brush is provided. In the example of FIG. 14A, the base-surface is rigid, but as shown below (FIG. 15D) in some embodiments, the base-surface may be conformable—e.g. having a first configuration (top of 15D. where the protrusions are parallel to each other) and a second configuration where due to conforming and/or base deformation that protrusions converge towards each other (bottom of FIG. 15D). The feature of FIG. 15D may allow the distal ends of the brush to conform to a shape of the scalp and the deformation of the base-surface may be in respond to higher pressure towards the center of the ‘field of protrusions.’

As shown in FIG. 14B, in some embodiments, it is possible to drag or ‘rake’ the brush across the user's scalp to obtain ‘streaks’ instead of the ion-deposition ions discussed above.

As shown in FIG. 15A-15C, it may be possible to provide a functionality similar to that of FIG. 15D (i.e. where the surface defined by the distal end of the protrusions ‘conforms’ to the scalp) by protrusion flexibility.

### Ion-Deposition Heterogeneity (e.g. of Deposition Islands)

FIG. 9 illustrates one island-deposition pattern where there is some degree of ‘deposition heterogeneity’ (e.g. in two dimensions as opposed to FIG. 6 which illustrates such heterogeneity only in a single dimension).

Towards this end, it may be useful for any embodiments (e.g. brush or roller) to distribute protrusions capable of depositing different types of ions over the ‘base’ surface of the roller or brush.

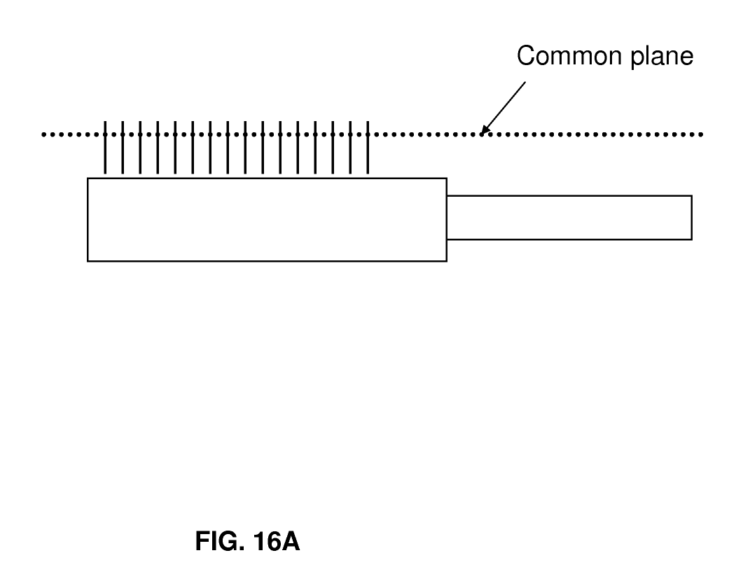

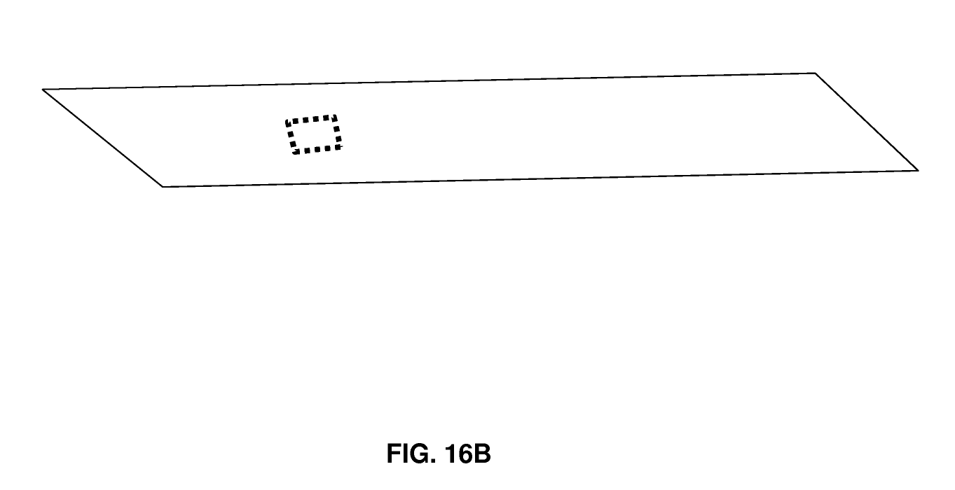

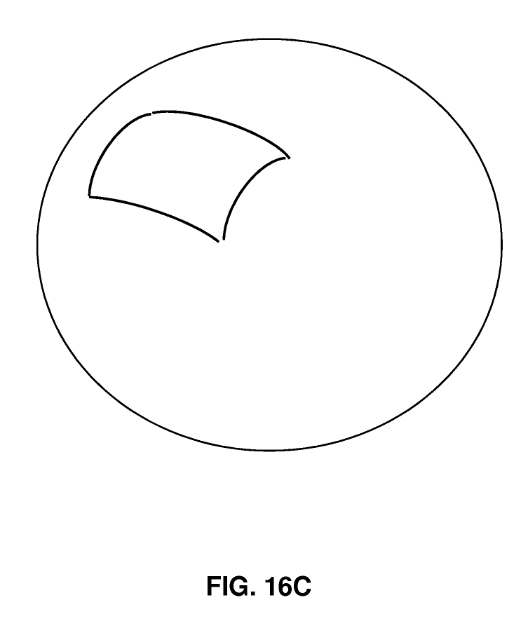

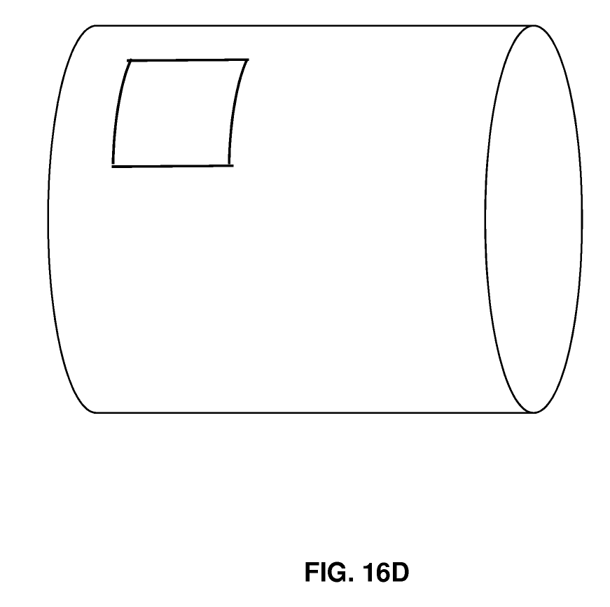

FIG. 16A illustrates a ‘common plane’ through which the protrusions pass. Just like it is possible to define the ‘heterogeneity’ in terms of island deposition on skin-patches, it is also possible to define ‘heterogeneity’ in terms of the capability of the ion deposition passing through a ‘patch’ of the ‘common plane.’ FIG. 16B illustrates a ‘patch’ for the device of FIG. 16B—for roller devices, the ‘square patch’ is a square in ‘curvilinear coordinates’ relative to a ‘round common-surface’ over the roller—this round common-surface′ has the same shape the roller surface (i.e. the round common-surface is a geometric construct and its shape is identical to that of the roller surface—if the roller surface is cylindrical than the round common-surface is cylindrical and if the roller surface is spherical than the round common-surface is spherical).

Examples of ‘square patches’ on the ‘curvilinear’ surface are illustrated in FIGS. 16C-16D.

### Light-Guide

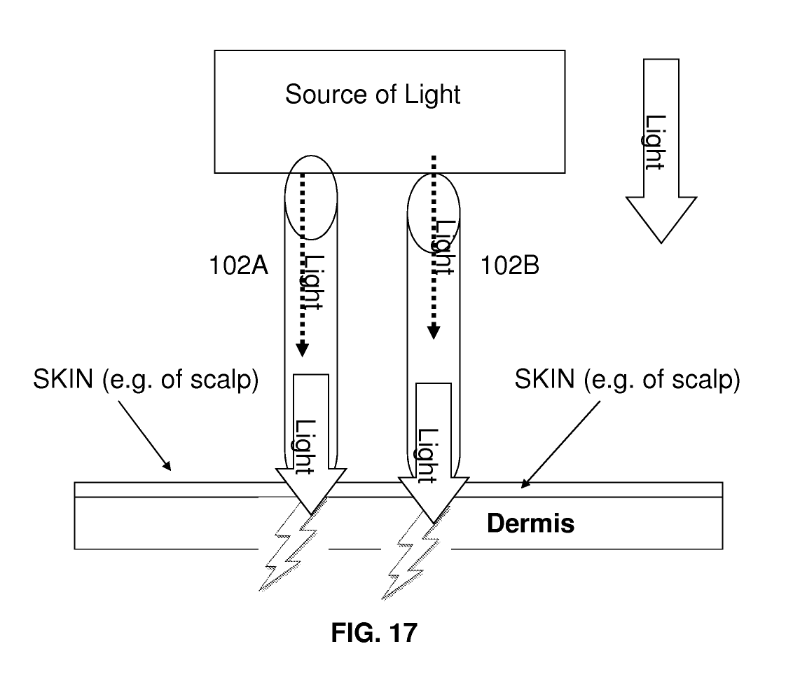

As shown in FIG. 17, in some embodiments, the electrode may be a ‘hybrid electrode protrusion’ for delivering both current (eg. at least partially ionic) as well as light (e.g. through a light-guide optical properties of the protrusion).

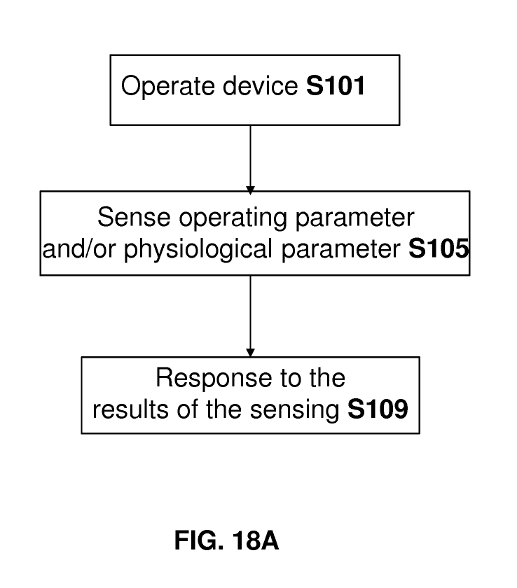

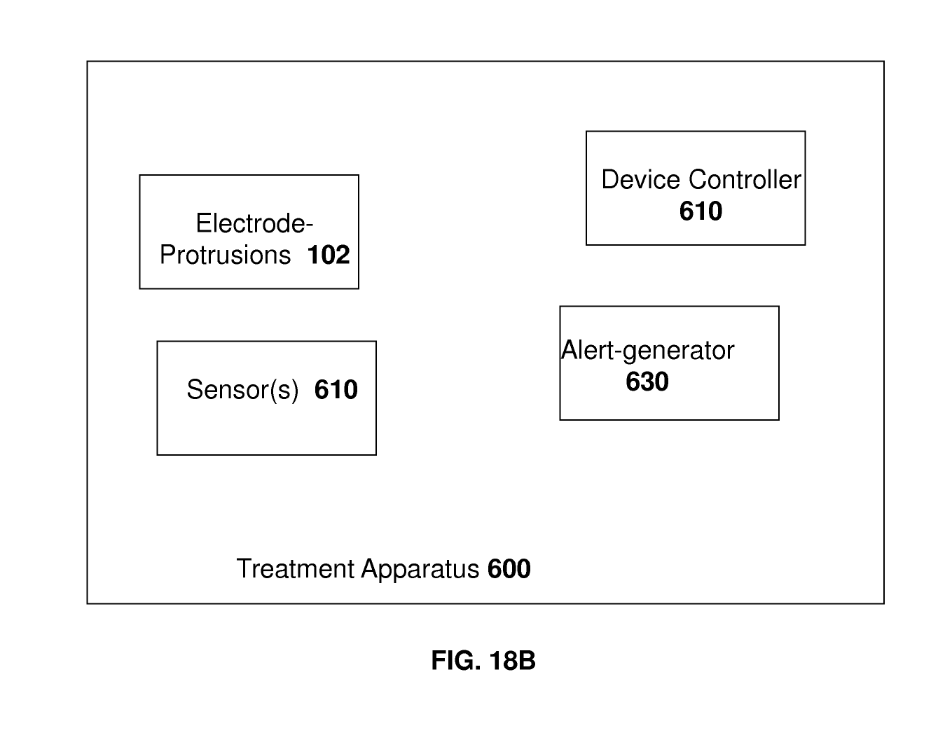

### Feedback—a Discussion of FIG. 18

FIG. 18A is a flow chart of a method for operating a device according to feedback. FIG. 18B is a block diagram of a system for performing the method of FIG. 18.

It may be possible (step S105) to measure an indication of how effective the treatment is—for example, to measure a rate at which ions are deposited on the scalp (greater deposition is more effective treatment) and/or an amount of current between electrodes (greater is more effective treatment) or a degree of color-change of the scalp (e.g. optically by a camera or in any other manner—greater change means irritation—more effective treatment).

If this indicator shows (step S109) that the treatment is not effective enough it is possible to generate an alert (e.g an ‘immediate alert’) to encourage the user, for example, to press harder on his/her scalp with the device.

Alternatively or additionally, the device may provide the user an indication of an end of a given treatment session—e.g. by an treatment-end alert signal (e.g. audio or visual or tactile) or by shutting off the vibration, current or light. In this case, if the indications shows the treatment is not effective enough it is possible to compensate by increasing a treatment duration—e.g. the amount of time which must elapse before the treatment-end indication (e.g. alert signal or shutting off) is provide to the user.

Alternatively or additionally, it is possible to compensate by increasing a voltage applied between electrode (e.g. ion-releasing electrodes). For example, it may be possible to respond with several voltage or current pulses (e.g. brief in duration—e.g. <1 sec or <0.5 sec or <0.1 sec or <0.05 sec)

For the first case (the ‘alert signal’), alert signal may be provided if the user is not pressing hard enough (even if painful)—this would encourage the user to press harder. For roller embodiments, the resistance to rolling may be increased if the user is not pressing hard enough.

In one embodiment, a ‘minimum treatment effectiveness’ is characterized by minimum current or ion-deposition rate or force or roll-rate (or any combination thereof).

In yet another embodiment, it is possible to optically and/or mechanically detect a presence of tangled or trapped hair (e.g. trapped in the roller or in any other device form-factor) and to respond with an alert signal.

In yet another embodiment, it is possible to regulate the ‘effective sharpness’ and/or ‘effective penetrating ability’ of the protrusion—e.g. by regulating the length of the mini-needle of FIG. 13—e.g. if the treatment effectiveness indicator is below a threshold, it may be possible to cause the protrusion to be ‘effectively sharper’ to compensate for too-little ion-deposition by greater penetration.

Alternatively or additionally, a scalp thickness sensor (e.g. based on ultrasound) may be provided—for thicker scalps it is possible to increase the intensity of treatment (e.g. effective sharpness and/or ion-deposition rate and/or voltage between electrodes).

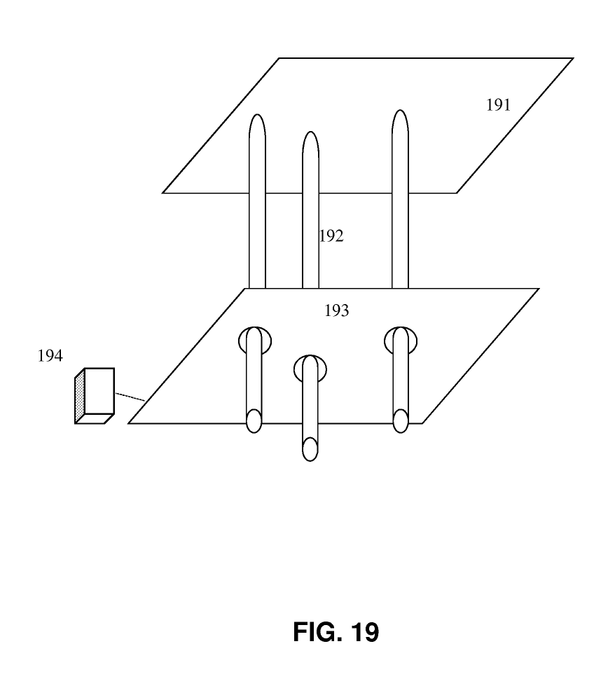

### FIG. 19

FIG. 19 describes one embodiment of a brush form with a vibrating plate.

In this embodiment, the brush comprises an optionally conforming base (191) and an optionally perforated vibration plate (193) the bristles (192) go through. The conformation of the base allows the bristles to move up and down through the perforate vibrating plate so a maximal number of bristles are in contact with the scalp simultaneously.

The vibration plate is located 5 mm, 10 mm, 15 mm, or 20 mm below the base and above the end of bristles. It is connected to a vibrator (194) that can vibrate it laterally. Optionally, the vibration is in each axis, optionally independently controlled per axis. This embodiment provides effective lateral vibration to the bristle ends, while adding configurable rigidity to the teeth. The distance between the base and vibrating plate determines the rigidity of the teeth.

Various embodiments and aspects of the present invention as delineated hereinabove and/or as claimed in the claims section below find experimental support in the following examples:

### EXAMPLE

### Experiment

Reference is now made to the following example, which together with the above descriptions illustrates some embodiments of the invention in a non-limiting fashion. In particular, features described below may be used without other described features and in conjunction with methods and/or apparatus as described above.

Material and Methods

An experiment over 2-4 months was conducted on 26 volunteers all of whom were suffering from baldness. Each volunteer was provided with a roller-like device (see, for example, FIG. 1) configured to form both zinc-ion-deposition islands and copper-ion-deposition islands when rolled over the scalp. As the user rolled the device over his respective scalp, electrodes of the roller device were each briefly brought into contact with and out of contact with the scalp. The device used included 8 disks with non-puncturing electrode protrusions, used for several minutes at least twice a week by users.

Each disk had about 100 protrusions, about 0.2 mm wide and a triangular protrusion with effective contact length of 1 mm (tip is about 0.1 mm)

Disks were alternatively coated with Zinc and Copper.

Electrical current applied was about 30V at 40 Hz.

No LLLT was applied.

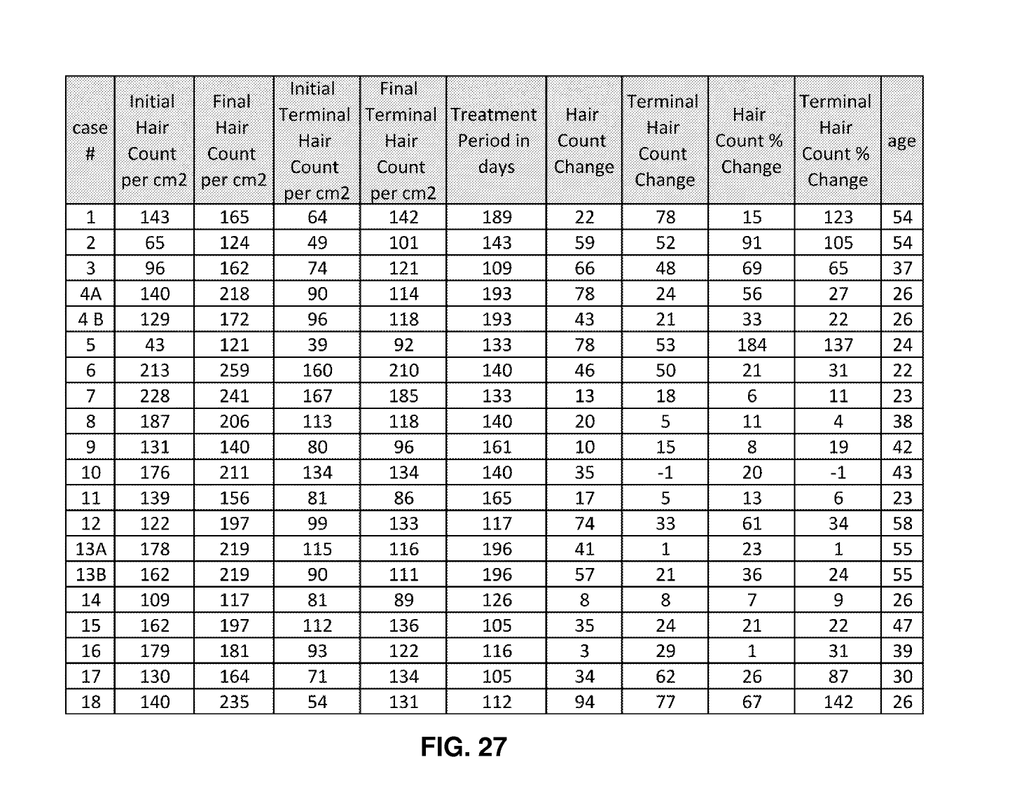

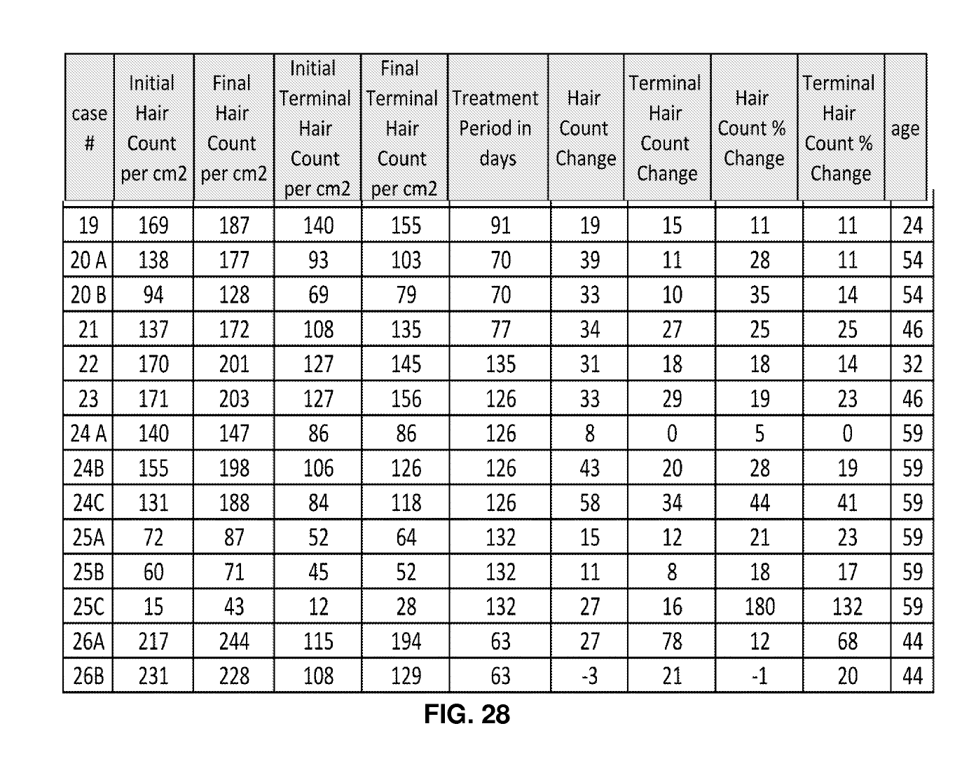

For each subject, it was possible, per treatment site, to monitor a number of features related to hair density at the treatment site, such as the overall hair density, terminal hair-density and non-terminal hair density. Results are summarized in FIGS. 27A-27B. The skilled artisan who reviews FIGS. 27A-27B will appreciate that the device and method appeared to play a significant roll in reversing hair-loss.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

It is expected that during the life of a patent maturing from this application many relevant hair stimulation devices will be developed and the scope of the term hair stimulation device is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

### APPENDIX

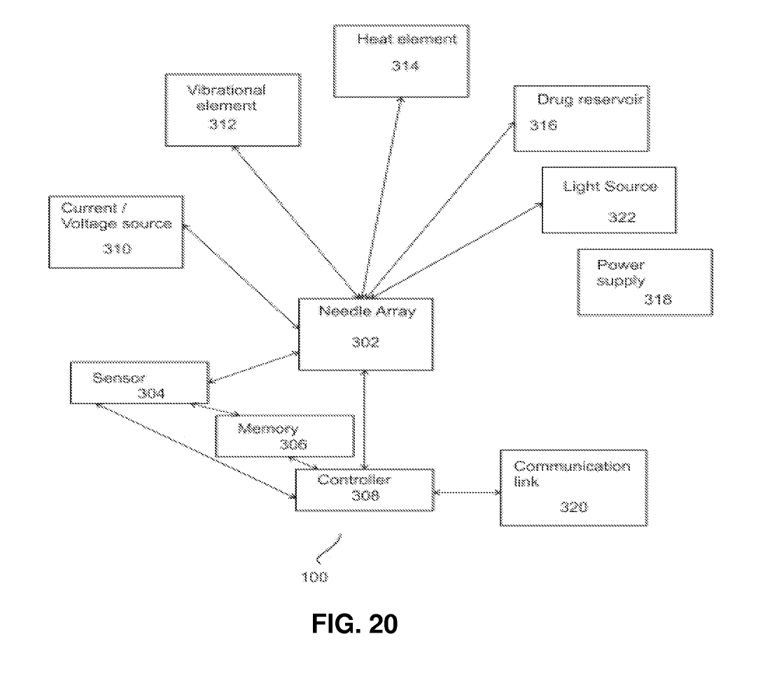

FIG. 20 is a general block diagram of the device, in accordance with an exemplary embodiment of the invention;

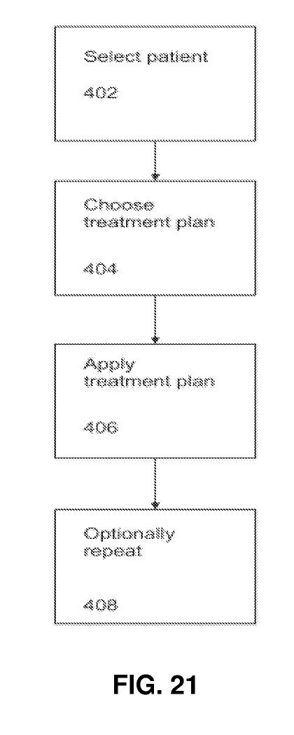

FIG. 21 is a flow chart of a method of stimulating hair growth, in accordance with an exemplary embodiment of the invention;

FIG. 21 is a flowchart of an exemplary method of stimulating scalp hair growth, in accordance with an exemplary embodiment of the invention. Optionally, the method uses the hair stimulation device.

Optionally, at 402, a patient is selected, in accordance with an exemplary embodiment of the invention. Optionally, the patient is male. Optionally or additionally, the patient has been diagnosed with androgenic alopecia. Optionally, the patient is at the early stages of hair loss (e.g., has not lost most of his hair)

Optionally, at 404, the treatment plan is selected, in accordance with an exemplary embodiment of the invention. Optionally, a mechanical stimulation protocol is selected. Optionally or additionally, a vibration stimulation protocol is selected. Optionally or additionally, a thermal stimulation protocol is selected. Optionally or additionally, an ion deposition protocol is selected. Optionally or additionally, an electrical stimulation protocol is selected.

In some embodiments of the invention, at least some of the stimulation protocols (e.g., vibration, thermal, ion, electrical) are applied substantially simultaneously. Alternatively or additionally, at least some of the protocols are applied successively, for example, in no particular order. Alternatively or additionally, some protocols are selectively applied, while other protocols are not applied.

In some embodiments of the invention, the treatment plan is selected manually, for example by a physician, for example, based on personal experienced and/or clinical guidelines. Alternatively or additionally, the treatment plan is selected automatically, for example by software, for example, based on collected experimental data.

In some embodiments of the invention, the treatment plan is selected over a long period of time, for example, a single treatment session is to be repeated for a duration of time. For example, a single treatment plan is repeated four times a day, three times a day, twice a day, once a day, every other day, three days a week, twice a week, once a week, or other smaller, intermediate or larger time frames and/or repetition rates are used. For example, treatment is repeated over a month, over two months, over six months, over one year, over two years, indefinitely, or other smaller or intermediate time frames are used. Optionally, treatment is stopped when a desired growth effect is achieved and/or a certain time after, for example, a week or a month. Optionally or alternatively, stimulation is stopped, or at least paused for a week or more, if further progress is not seen. Optionally, the application and/or delay of treatment depends on scalp thickness, with treatment, for example, being continued as long as scalp thickness continues to increase and/or only if an increase is found.

In an exemplary embodiment of the invention, a maintenance level of treatment is defined and followed by the user.

In some embodiments of the invention, the treatment plan is selected so that a different part of the scalp is treated during different treatments. For example, treatment may be twice a day with a different part of the scalp treated during each of the two daily treatments. Optionally, the areas of treatment during different treatment sessions partially overlap.

In some embodiments of the invention, the time per session is selected. For example, about 30 seconds, 1 minute, 2, 4, 6, 10 minutes, or other smaller, intermediate or larger times or subranges thereof are used. Optionally, the time is selected according to a pain level caused by the device and/or a user pain and/or comfort threshold.

In some embodiments of the invention, the treatment area is selected. For example, approximately 50% of the total area in need of treatment, 10%, 25%, 33%, 67%, 75%, 90%, 100% or other smaller, intermediate or larger areas or subranges thereof are used.

At 406, the treatment plan and/or protocol is applied to the patient, in accordance with an exemplary embodiment of the invention. For example, the patient holds the device, and rolls the discs over the area of his scalp that requires stimulation. The needles on the discs prick his scalp according to the mechanical stimulation protocol. Optionally or additionally, the needles are vibrated according to the vibration protocol. Optionally or additionally, the skin is heated underneath the surface (e.g., heat transferred through the needles) according to the thermal stimulation protocol. Optionally or additionally, ions are deposited into below the skin (e.g., released from metallic coating on the needles) according to the ion deposition protocol. Optionally or additionally, electrical current and/or voltages are applied underneath the surface of the skin (e.g., using the needles as electrodes) according to the electrical stimulation protocol.

In a non-limiting example, a protocol comprises of treatments applied 3 times a week, for about 5 minutes per treatment. Each treatment comprises the following stimulations: 5 Volts, at 100 Hz AC, Zinc biased duty cycle, heating to a temperature of 60 degrees Celsius and vibration. Optionally, the protocol is selected according to trial and error, for example, the protocol is adjusted after a couple of weeks depending on the response of the scalp.

Optionally, at 408, the treatment is repeated, for example, according to the plan as in 404, in accordance with an exemplary embodiment of the invention. Optionally, the same treatment protocol is repeated. Alternatively, the treatment protocol is adjusted. For example, the initial treatment protocol is selected, the treatment is applied, and the treatment is adjusted based on feedback of success of the treatment.

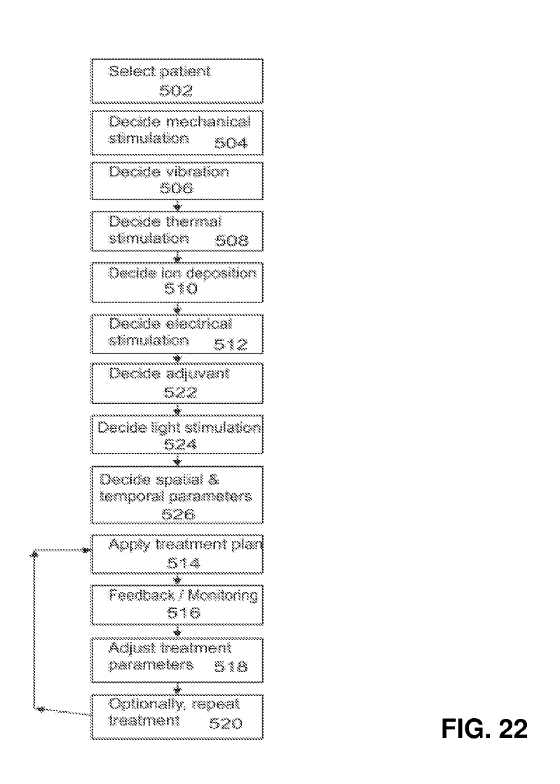

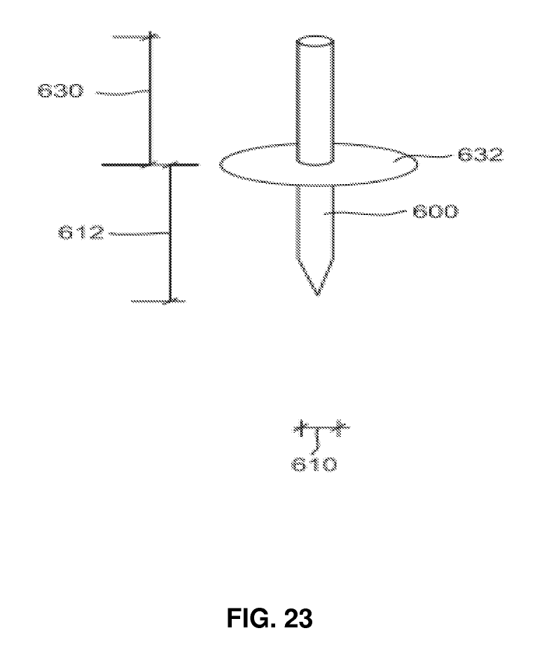

FIG. 22 is a flowchart of a detailed method of FIG. 21, in accordance with an exemplary embodiment of the invention;

### Exemplary Method of Treatment

FIG. 22 is a detailed method of treatment of FIG. 104, in accordance with an exemplary embodiment of the invention.

Optionally, at 502, a patient is selected for treatment.

Optionally, at 504, a decision is made with regards to the mechanical stimulation protocol.

Optionally, at 506 a decision is made with regards to the vibration protocol.

Optionally, at 508 a decision is made with regards to the thermal stimulation protocol.

Optionally, at 510 a decision is made with regards to the ion application protocol.

Optionally, at 512 a decision is made with regards to the electrical stimulation protocol.

Optionally, at 522 a decision is made with regards to the use of adjuvant treatment.

Optionally, at 524 a decision is made with regards to the use of light stimulation.

Optionally, at 526 a decision is made with regards to the spatial and temporal parameters.

Optionally, at least one of the parameters chosen in steps 504, 506, 508, 510, 512, 522 and 524 are specific per scalp area and are determined individually for each scalp area to be treated. For example, the temple area could receive one treatment and the vertex area could receive a different treatment. For example, it may be determined to treat the vertex area consecutively 5 minutes daily while the temples area is to be treated consecutively 4 minutes daily.

At 514, the treatment plan is applied.

Optionally, at 516 feedback related to the treatment is obtained.

Optionally, at 518 one or more variables of one or more treatment protocols are adjusted. Optionally, the adjustment is related to the feedback as in 516.

Optionally, at 520 treatment is repeated.

Per FIG. 6A, in an exemplary embodiment of the invention, needles, for example needles 600, are selected and/or arranged as an array according to the selected mechanical stimulation protocol. Non-limiting examples include; a cross sectional diameter 610 corresponding to the selected area of individual contacts and/or penetrations, a length 612 corresponding to the selected depth of pnetration (optionally, a stopper 632, for example a flat disc, is used to set the needle length to prevent the needle from deeper penetration into the skin).

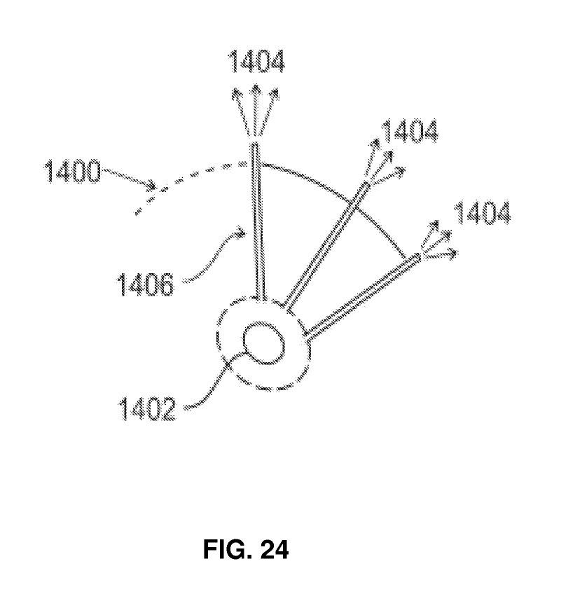

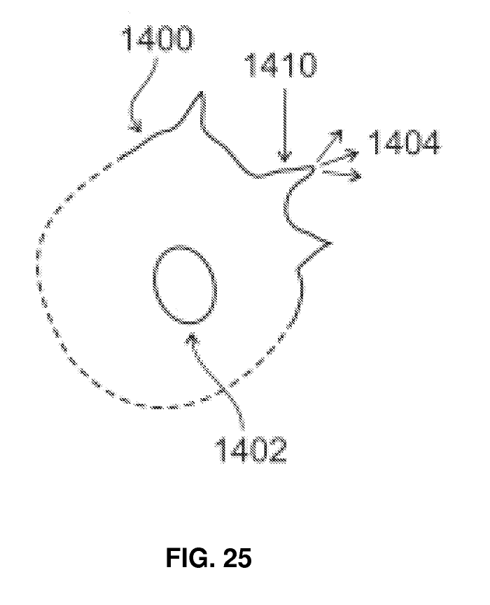

FIGS. 25 and 24 illustrate discs comprising a light source. In an exemplary embodiment, light conducting disc 1400 (FIG. 25) comprises light source 1402 causing light 1404 to emanate from spike 1410 on disc 1400. Optionally, disc 1400 comprises translucent material. Optionally or alternatively, spike 1410 comes to a sharp point. Optionally, spike 1410 is metallic.

FIG. 14B illustrates an exemplary embodiment in which light 1404 originates from light source 1402 and travels through optical fibers 1406 embedded in disc 1400. Optionally, the optical fibers 1406 penetrate directly into the skin. Optionally, optical fibers 1406 are thin enough to easily penetrate skin.

In some embodiments, one or more discs each comprise multiple fibers and/or needles. Optionally, at least one disc is for optical stimulation. Optionally or alternatively, at least one disc is metallic. Optionally or alternatively, at least one disc includes both optical needles and metallic needles. Optionally, at least one needle is both optical and metallic. Optionally or alternatively, fiber and/or needle are provided on parallel discs. Optionally or alternatively, fibers and/or needles are provided in a planar array.

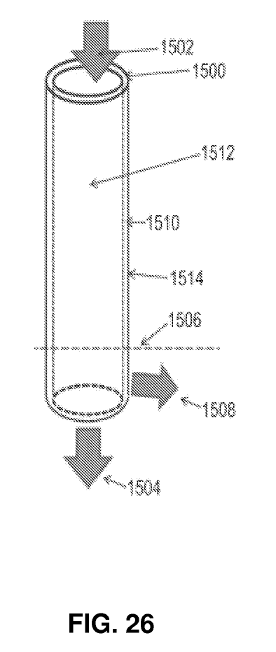

FIG. 26 illustrates an injector comprising a light guide, in accordance with an exemplary embodiment of the invention. In an exemplary embodiment, the light guide is an optical fiber coated with metal. For example, light is produced by light source 1502 which is powered by power source 1500 and emanates light 1504. Optionally, power source 1500 is electrical.

In some embodiments, power source 1500 emits ions 1508 directly into the scalp beneath the scalp surface 1506. Optionally, power source 1500 emits electricity directly into the scalp beneath the scalp surface 1506. Optionally, power source 1500 emits heat directly into the scalp beneath the scalp surface 1506. Optionally, the discs, needles and/or optical fibers also vibrate.

In some embodiments, the injector comprises a cavity 1512. Optionally, cavity 1512 comprises a light conducting core. For example, cavity 1512 may comprise light transmitting material. Optionally, the light transmitting material has structural rigidity. Optionally or alternatively, the light transmitting material has minimal structural rigidity.

In some embodiments, cavity 1512 comprises an internal optical fiber. For example, the internal optical fiber may comprise a metal coated thin optical fiber. Optionally or alternatively, the internal optical fiber may comprise an external shell conducting electricity. Optionally or alternatively, the internal optical fiber may comprise an external shell conducting heat. Optionally or alternatively, the internal optical fiber may comprise an external shell conducting injecting ions into the skin. Optionally or alternatively, the internal optical fiber may emit light into the skin.

In some embodiments, hollow cavity 1512 comprises a void which transmits light. Optionally, the outer portion 1510, inside outer layer 1512, of the injector comprises a source of vibration. Optionally or alternatively, the outer portion of the injector comprises a source of heat.

In some embodiments, the outer layer 1514 comprises an electrical conductor. For example, outer layer 1514 comprises metal. Optionally, outer layer 1514 is coated with ions to be deposited. For example, outer layer 1514 is coated with Cu. Alternatively, outer layer 1514 is coated with Zn. Optionally, outer layer 1514 comprises heat conducting material.

### General

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

1. (canceled)

2. Apparatus for treating the scalp comprising:
a plurality of ion-releasing electrode protrusions configured so that when first and second of the protrusions are simultaneously in contact with human skin, at least partially-ionic current flows between the first and second electrode protrusions via the skin so as to deposit on the skin, a ion and a counter-ion thereof, the ion and counter-ion being released from the first and/or second electrode protrusions.

3. The apparatus of claim 2 wherein the plurality comprises at least 2 or at least 5 or at least 10 or at least 20 or at least 30 or at least 50 or at least 75 or at least 100 or at least 150 or at least 200 or at least 300 protrusions.

4. The apparatus of claim 3 wherein each electrode of the plurality is respectively associated with a respective counter-electrode of the electrode plurality, optionally a lateral displacement between the electrode and its respective counter-electrode being at most 1 cm or at most 7.5 mm or at most 5 mm, to define a respective electrode-pair such that when both electrodes of the respective electrode-pair are simultaneously in contact with human skin, at least partially-ionic current flows between the electrode protrusions of the respective electrode-pair via the skin so as to deposit ions, released from any one or both electrodes of the respective electrode-pair on the skin.

5. The apparatus of claim 3 wherein each electrode of the plurality is respectively associated with a respective counter-electrode of the electrode plurality, optionally a lateral displacement between the electrode and its respective counter-electrode being at most 1 cm or at most 7.5 mm or at most 5 mm, to define a respective electrode-pair such that when both electrodes of the respective electrode-pair are simultaneously in contact with human skin, at least partially-ionic current flows between the electrode protrusions of the respective electrode-pair via the skin so as to deposit at least two counter-ions, released from any one or both electrodes of the respective electrode-pair on the skin.

6. The apparatus of claim 2 wherein each electrode of the plurality is operatively coupled to mechanical actuator(s) configured to repeatedly bring pairs of the electrode into and out of contact with a surface of skin so as to form ion-deposition islands thereon.

7. The apparatus of claim 2 wherein each electrode of the protrusion extends from a base-surface selected from the group consisting of (i) a surface of a wheel or cylindrical roller or spherical roller or disc; (ii) a rigid flat surface; and (iii) a conformable surface that is-flat in at least one configuration.

8. The apparatus of claim 7 wherein the roller is a cylindrical roller having a limited roll-range, for example, at most 270 degrees or at most 180 degrees or at most 135 degrees or at most 90 degrees or at most 60 degrees or at most 45 degrees.

9. The apparatus of claim 2 wherein a separation distance between the first and second protrusions is at most 1 cm or at most 5 mm.

10-23. (canceled)

24. A scalp-treatment apparatus comprising:
a. a disc-array of at least two or at least 3 or at least 4 or at least 5 or at least 10 co-axial discs (e.g. thin discs) spaced along a roller central axis;
b. ion-releasing electrode-protrusions being disposed around a circumference of each of the discs and radially protruding therefrom such that when first and second of the protrusions are simultaneously in contact with human skin, at least partially-ionic current flows between the first and second electrode protrusions via the skin so as to deposit on the skin, a ion and a counter-ion thereof, the ion and counter-ion being released from the first and/or second electrode protrusions.

25. The apparatus of claim 24 wherein the roller is continuous along its central axis.

26. The apparatus of claim 24 wherein the roller comprises a disc-array of at least two or at least 3 or at least 4 or at least 5 or at least 10 thin co-axial discs spaced along a roller central axis, the electrode-protrusions being disposed around a circumference of each of the discs and radially protruding therefrom.

27. The apparatus of claim 25 wherein a thickness of the each thin-disc is 0.75 mm or at most 0.5 mm or at most 0.25 mm or at most 0.1 mm.

28. The apparatus of claim 24 wherein all discs of the disc-array rotate in-tandem with each other.

29. The apparatus of claim 24 wherein for each pair of neighboring discs, inter-disc distance therebetween along the roller central axis (i) at least 2 mm and/or (ii) at most 1 cm or at most 8 cm at most 6 mm and/or (iii) at least 5 times or at least 10 times or at least 20 time a thickness of a thickest disc of the disc-array.

30. The apparatus of claim 24 wherein each disc has a diameter of at least 10 mm, or at least 20 mm, or at least 30 mm, or at least 40 mm, or at least 50 mm, or at least 60 mm, or at least 70 mm.

31. The apparatus of claim 24 wherein configured so that for at least one pair of neighboring discs, an annular portion of an inter-disc region therebetween is substantially void, wherein (i) a length of the annular portion is at 20% or at least 30% or at least 40% or at least 50% an inter-disc distance between the neighboring discs; and (ii) an outer diameter of the annular portion is at least that of the neighboring discs; and (iii) an inner diameter of the annular portion is at most 5 mm or at most 10 mm less than that the of the neighboring discs.

32. The apparatus of claim 24 where there is a round-roller (e.g. cylinder but not only) (i) comprising the cylindrical roller and/or (ii) wherein the one or more discs along the common rotation axes so that outer diameters thereof substantially lie along a common geometrical cylinder, thereby defining a cylindrical roller, wherein the cylindrical roller has a limited roll-range, for example, at most 270 degrees or at most 180 degrees or at most 135 degrees or at most 90 degrees or at most 60 degrees or at most 45 degrees.

33. The apparatus of claim 24 further comprising (i) at least sensor configured to sense at least one parameter related to operation of the apparatus and/or to a status of skin treated by the apparatus; and (ii) at least one response-element configured to generate a response, responsively to the results of the sensing.

34. The apparatus of claim 32 wherein at least one sensor(s) is selected from the group consisting of:
i. an ion-deposition rate sensor configured to sense a rate of deposition of ions on the skin by the at least partially-ionic current;
ii. a force or pressure sensor configured to sense an amount of force or pressure between the electrode-protrusion(s) and skin;
iii. skin-color sensor;
iv. a current sensor configured to sense a magnitude of current via the skin via electrode-protrusions;
v. a trapped or tangled hair sensor configured to sense a presence or absence or amount of hair trapped within or entangled to the roller (e.g. mechanical and/or optical);
vi. a skin wetness sensor;
vII. a skin temperature sensor (e.g. based on IR); and
vIII. a scalp thickness sensor (e.g. based on ultrasound).
IX. ROLL COUNTER
X. ACCELEROMETER

35-36. (canceled)

37. A device comprising:
a plurality of electrode protrusions configured, when at least two of the protrusions are simultaneously in contact with human skin, at least partially-ionic electric current flows between first and second protrusions via the skin,
wherein the protrusions are disposed around the circumference of a wheel or roller having only partial rotational freedom.

38-73. (canceled)
